(19) United States
(12) Patent Application Publication
(10) Pub. No.: US 2013/0324561 A1
(43) Pub. Date: Dec. 5, 2013

(54) ATROPISOMERS OF P13K-INHIBITING COMPOUNDS

(71) Applicants: Jerry Evarts, Redmond, WA (US); Roger G. Ulrich, Seattle, WA (US)

(72) Inventors: Jerry Evarts, Redmond, WA (US); Roger G. Ulrich, Seattle, WA (US)

(73) Assignee: GILEAD CALISTROGA LLC., FOSTER CITY (CA)

(21) Appl. No.: 13/825,747

(22) PCT Filed: Sep. 23, 2011
(86) PCT No.: PCT/US11/53102

§ 371 Date: Jun. 5, 2013

Related U.S. Application Data
(60) Provisional application No. 61/386,420, filed on Sep. 24, 2010.

(51) Int. Cl. C07D 487/04; C07D 473/00

(52) U.S. Cl. CPC C07D 487/04; C07D 473/00
USPC 514/263.2; 544/262; 514/262.1; 544/277

(57) ABSTRACT

The invention provides compounds, compositions and methods to treat certain inflammatory conditions or cancers by administering a compound that inhibits PI3K isoforms, wherein the compounds are optically active atropisomers. It provides optically active stereoisomers of a class of pyrazole-containing compounds, which are useful for these methods, and provides methods to obtain these compounds as well as pharmaceutical compositions containing these compounds.

### RELATED APPLICATIONS

This application claims benefit of U.S. application Ser. No. 61/386,420 filed Sep. 24, 2010, which is incorporated herein by reference in its entirety.

### TECHNICAL FIELD

The invention is in the field of therapeutics and medicinal chemistry for the treatment of medical conditions using compounds that inhibit phosphatidylinositol-3-kinases (PI3Ks), particularly the delta isoform of PI3K (PIKδ) enzymes in vivo. In particular, the invention concerns compounds, compositions, and methods of treatment of inflammatory conditions with enantiomerically enriched atropisomers of substituted pyrazole compounds.

### BACKGROUND ART

Inflammatory responses may result from infection by pathogenic organisms and viruses, noninfectious means such as trauma or reperfusion following myocardial infarction or stroke, immune responses to foreign antigens, and autoimmune diseases. Inflammatory responses are notably associated with the influx of leukocytes and/or leukocyte chemotaxis. Leukocytes provide a first line of immune defense against many common microorganisms.

Identification of the delta (δ) isoform of phosphatidylinositol 3-kinases (PI 3-kinases; PI3Ks) is described in Chantry, et al., J Biol Chem (1997) 272:19236-19241. It was observed that the human p110δ isoform is expressed in a tissue-restricted fashion. It is expressed at high levels in lymphocytes and lymphoid tissues, suggesting that the protein might play a role in PI 3-kinase-mediated signaling in the immune system.

PI3Kδ inhibitors are useful to treat inflammatory conditions. For example, Lee, et al., FASEB J. (2006) 2:455-465, describes evidence that inhibition of PI3Kδ attenuates allergic airway inflammation and hyperresponsiveness in murine asthma models, demonstrating that selective inhibitors of PI3Kδ are useful to treat asthma and allergic reactions as well as immune disorders.

PI3Kδ inhibitors are also useful for treating other medical conditions, including certain cancers. Flinn, et al., J. Clin. Oncol. 27:15s, 2009 (suppl; abstr 3543). There is a need for selective inhibitors of PI3K and of PI3Kδ inhibitors in particular for the treatment of diseases and conditions mediated by excessive activity of PI3K.

### DISCLOSURE OF THE INVENTION

The invention relates to selective PI3Kδ inhibitors and methods to treat inflammatory conditions and cell proliferative disorders with compounds that are selective inhibitors and particularly with selective inhibitors of PI3Kδ. Compounds of the invention are prepared as optically active atropisomers, where the separated atropisomers have unexpected advantages over mixtures of atropisomers for use in treatment of inflammation or cancer. The compounds, compositions, and methods of the invention are therapeutically beneficial in treating conditions associated with excessive or undesired levels of PI3K activity.

In one aspect, the invention provides an optically active compound enriched in one of the atropisomers of formula 1:

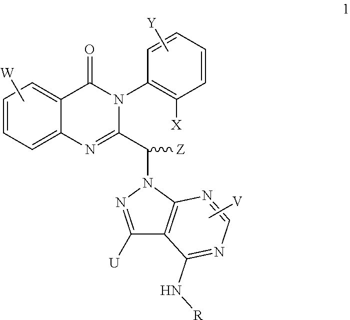

wherein W, Y, and V are each independently H, halo, R¹, OR¹, CF₃, CN, where each R¹ is independently C1-C4 alkyl;

A is CH or N;

X is selected from Cl, Br, Me, CF₃, CN, and OMe;

Z is H, Me, Et, n-Pr, or cyclopropyl;

R is H or C1-C4 acyl; and

U is selected from aryl, heteroaryl, alkenyl, and alkynyl, each of which is optionally substituted;

or a pharmaceutically acceptable salt thereof.

In one embodiment, the invention provides an optically active compound comprising an atropisomer of formula 1(S)

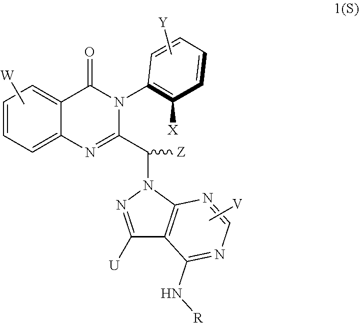

wherein A, U, V, W, X, Y, Z and R are as defined for formula 1; and wherein the atropisomer of formula 1(S) is present in excess of its corresponding enantiomer of formula 1(R)

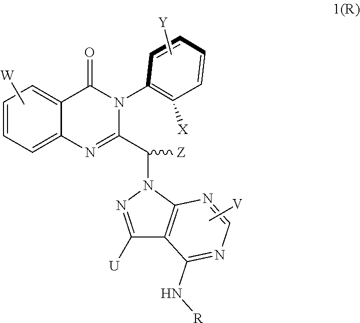

wherein A, U, V, W, X, Y, Z and R are as defined identically to formula 1(S), or a pharmaceutically acceptable salt of such compound. In some embodiments the compound consists of a single atropisomer; in some embodiments it consists essentially of one atropisomer. In specific embodiments, X is selected from the group consisting of Cl, Me and OMe; R is H; and W, Y, and V are independently selected from H, halo, Me, and OMe; Z is H, Me or Et; and U is aryl or alkynyl, each of which is optionally substituted.

Some examples of compounds of this type have been described in Chemistry & Biology, vol. 12, 123-34 (2010), but the occurrence of separate atropisomers, or their stability as individual atropisomers under physiological conditions were not disclosed. Moreover, the advantages that can be provided by one atropisomer over the other atropisomer or over a mixture of atropisomers have not heretofore been recognized.

The present invention relates to the separated atropisomers of compounds of formula 1, and selected subclasses, such as compounds of formula 2. In these compounds, U and R are as defined for formula 1.

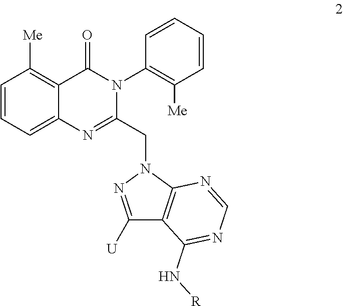

Similar-looking compounds with activity against PI3K are described in U.S. Pat. No. 6,800,620, to Sadhu, et al. A patent application describing selected atropisomers of one of the compounds in that patent has been filed as U.S. patent application Ser. No. 12/731,089.

In another aspect, the invention provides an optically active atropisomer of formula 1(R)

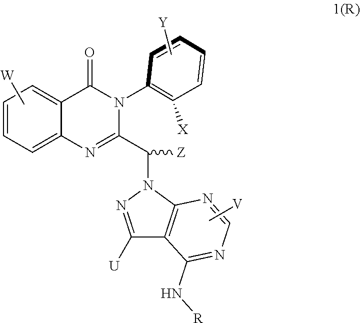

wherein U, V, W, X, Y, Z and R¹ are as defined for formula 1 in excess of its corresponding enantiomer of formula 1(S)

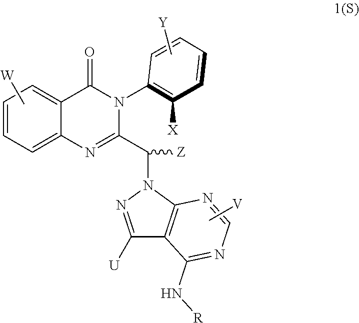

wherein U, V, W, X, Y, Z and R are as defined for formula 1(R);

or a pharmaceutically acceptable salt of such compound. In some embodiments the compound is a single atropisomer, and the opposite atropisomer is absent; in some embodiments it consists essentially of one atropisomer. In specific embodiments, X is selected from the group consisting of Cl, Me and OMe; R is H; and W, Y, and V are independently selected from H, halo, Me, and OMe; Z is H, Me or Et; and U is aryl or alkynyl, each of which is optionally substituted.

In yet another aspect, the invention provides an optically active compound of formula 1 that is a compound of formula 2 as described herein. In certain embodiments, this compound is an atropisomer of formula 2(S)

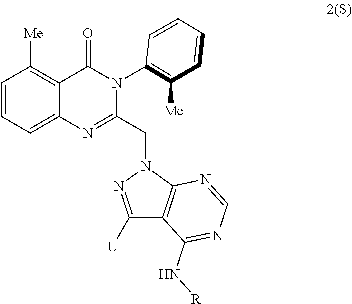

or a pharmaceutically acceptable salt thereof; and wherein the atropisomer of formula 2(S) is present in excess of its corresponding enantiomer of formula 2(R). In these compounds, U and R are as defined for formula 1.

In an alternative embodiment, this compound is an optically active atropisomer of formula 2(R)

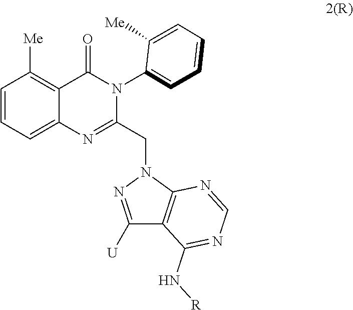

or a pharmaceutically acceptable salt thereof; and wherein the atropisomer of formula 2(R) is present in excess of its corresponding enantiomer of formula 2(S). In these compounds, U and R are as defined for formula 1. In some embodiments the compound is a single atropisomer (the other atropisomer is absent); in some embodiments it consists essentially of one atropisomer.

In another aspect, the invention provides an optically active atropisomeric compound of formula 5:

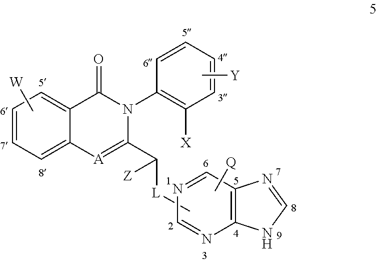

wherein W is an optional substituent that can be halo, C1-C4 alkyl, C1-C4 alkoxy, or CF₃;

Z can be H or C1-C4 alkyl, or if L is NR², Z and N can be linked together to form a 5-6 membered optionally substituted ring;

A is CH or N;

L can be NR² or S or a bond, and can be attached to position 6 or 9 of the purine ring;

Q can be H, Me, OMe, halo, or NH₂ or U, and is attached to the purine at position 2, 6, or 8 if L is attached at position 9, or at position 2 or 8 if L is attached at position 6;
U can be aryl, heteroaryl, cycloalkyl, heterocycloalkyl, alkyl, alkenyl, or alkynyl, each of which is optionally substituted;

X is Me, CF₃, Cl, CN, or Br;

Y can be H, C1-C4 alkyl, halo, CF₃, OMe, OH, NH₂, NHAc, or CN;

with the proviso that W and X are not both Me when Z is H, Q is NH₂, W is at position 5′ and L is a bond;

or a pharmaceutically acceptable salt thereof.

In some embodiments the compound is a single atropisomer; in some embodiments it consists essentially of one atropisomer. A compound present in excess of its opposite atropisomer thus typically contains less than 10% of the opposite atropisomer, and may be entirely free of the opposite atropisomer.

The optically active atropisomeric compounds are made and used as a single atropisomer, or as predominantly one atropisomer with less than 10% of the opposite atropisomer, and preferably less than 5% of the opposite atropisomer. Similar compounds are described in WO 2009/088990A1, but their stable atropisomerism and the advantages of isolating and using a single atropisomer do not appear to have been recognized.

In these compounds, X is a substituent that is large enough to induce atropisomers formed by hindered rotation about the N-phenyl(X) bond to be stable and separable, providing a center of chirality. If Z is not H, the compounds can also contain a chiral center where Z is attached; optionally, the compounds can be a singe isomer at this chiral center, e.g., at least about 90% and preferably 95% or more one enantiomer at this chiral center. In preferred embodiments, the chiral center bearing Z is predominantly in the S configuration. Applicants have found that the selection of a single atropisomer in combination with the selection of the S enantiomer at the chiral center bearing Z obviates complex separation and purification problems, and also substantially pharmacokinetic variations that would arise from use of compounds containing mixtures of diastereomers, while preserving the most desirable isomer and removing material that differs in important pharmacokinetic respects from the preferred isomer.

Some preferred embodiments of these compounds include compounds of formula 5a and 5b, wherein A, Q, W, X, Y, Z and L are as defined for formula 5:

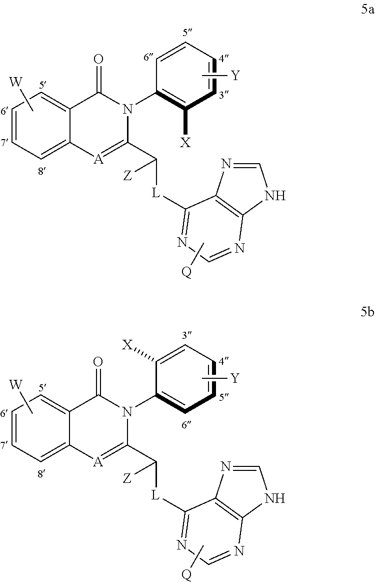

and compounds of formula 6a and 6b, where A, Q, W, X, Y, Z and L are as defined for formula 5:

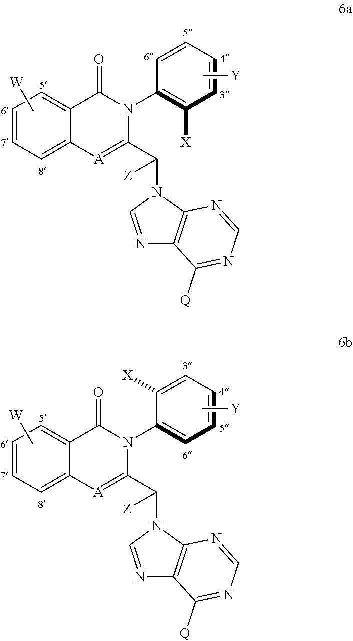

In these compounds, optical activity arises because one atropisomer is present in excess over the other, and the two atropisomers are stable to rotational interconversion at room temperature. Rotational interconversion is inhibited because X is large enough to hinder rotation about the phenyl-N bond. In these compounds, X is often Me; W is often Me, F or C, and is preferably at position 5′ or 6′; Y may be H; and Z can be H, Me, or Et. Frequently Q is H in formula 5a or 5b, and Q is H or NH, in formula 6a or 6b. A can be CH or it can be N in these compounds. Combinations of these preferred components provide preferred compounds.

In another aspect, the invention provides a composition comprising an optically active compound described herein, admixed with a pharmaceutically acceptable carrier and optionally an additional pharmaceutically acceptable excipient. The optically active compound contains one atropisomer of any of the foregoing structures in excess over its opposite atropisomer, and may contain only one atropisomer or may consist essentially of one atropisomer. These optically active compounds may also have an additional chiral center when Z is not H, and may be obtained and used as a single enantiomer or may consist essentially of a single enantiomer at the additional chiral center.

The invention also provides compounds of formula 1, 1(S), 1(R), 2, 2(S), 2(R), 5, 5a, 5b, 6a or 6b, in which from 1 to n hydrogens attached to a carbon atom is/are replaced by deuterium, in which n is the number of hydrogens in the molecule. Such compounds exhibit increased resistance to metabolism, and are thus useful for increasing the half life of any compound of formula I when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

In another aspect, the invention provides a method of treating a medical condition in a mammal, wherein the condition is characterized by inflammation. The method comprises administering to a mammal in need thereof an effective amount of an optically active atropisomeric compound described herein. The compounds of the invention inhibit PI3K delta and/or PI3K gamma, preferably inhibiting one or both of these isoforms more strongly than they inhibit other isoforms of PI3K. In certain embodiments, the inhibitors have a gamma/delta IC50 ratio of less than about 10, and preferably an IC50 less than about 250 nM on the delta isoform.

In some embodiments, the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury; which comprises administering to said mammal a therapeutically effective amount of an optically active atropisomer described herein.

In another aspect, the invention provides an optically active atropisomer obtained by chiral chromatographic separation of an enantiomeric mixture, or racemic mixture, of formula 1 or of formula 5 as described herein.

In some embodiments, an enantiomeric mixture of formula 1, e.g., a racemic mixture, is separated using a normal phase chiral column, and two peaks, A and B, are resolved, wherein peak A and peak B represent the atropisomers, 1(S) and 1(R), respectively,

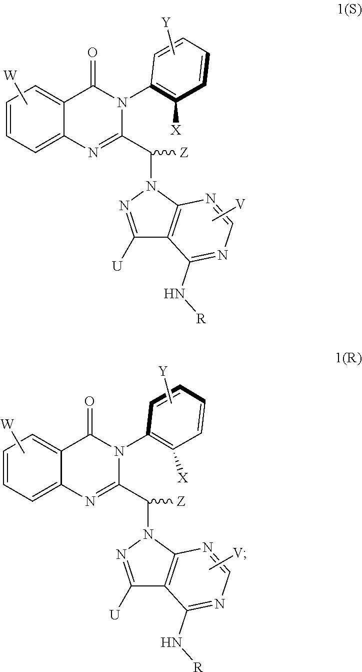

and

wherein the predominant optically active atropisomer obtained is the first compound to elute from the column.

In yet another aspect, the invention provides an optically active atropisomer obtained by chiral chromatographic separation of an enantiomeric mixture, or racemic mixture, of formula 2

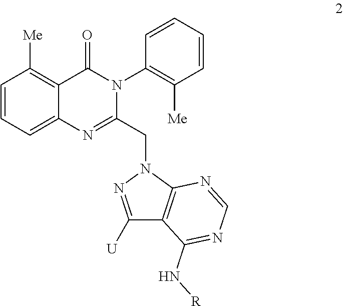

wherein U is as defined above for formula 1, or a pharmaceutically acceptable salt thereof; wherein an enantiomeric mixture of formula 2 is separated using a normal phase chiral column, and two peaks, A and B, are resolved, wherein peak A and peak B represent the atropisomers, 2(S) and 2(R), respectively,

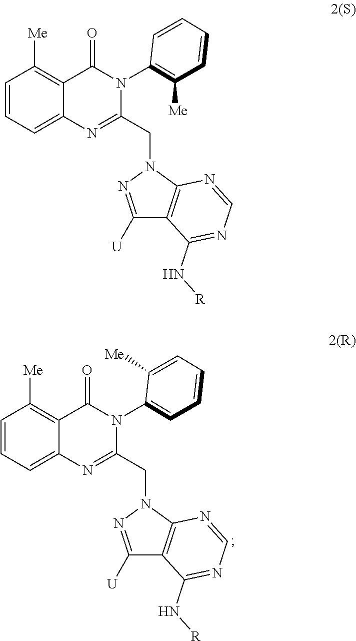

wherein the predominant optically active atropisomer to be used is the second compound to elute from the column.

Similarly, the invention provides an optically active atropisomer of a compound of formula 5, 5a, 5b, 6a or 6b obtained by chromatographic separation of a mixture (e.g., a racemic mixture) of the atropisomeric forms of the compound.

In another aspect, the invention provides pharmaceutical compositions comprising any of the optically active compounds described herein, and at least one pharmaceutically acceptable excipient, and optionally at least two different pharmaceutically acceptable excipients. Additional aspects and embodiments are provided in the detailed description below.

### DETAILED DESCRIPTION

Many organic compounds exist in optically active forms, i.e., they have the ability to rotate plane-polarized light. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are mirror images of one another. Stereoisomers that are mirror images of one another may also be referred to as enantiomers, and a mixture of such isomers is often called an enantiomeric mixture. A 50:50 mixture of enantiomers is referred to as a racemic mixture or a racemate. The terms “racemic mixture” and “racemate” refer to an equimolar mixture of two enantiomeric species, which is devoid of optical activity.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

The term “enantiomers” as used herein, refers to two stereoisomers of a compound.

The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical, i.e., optical activity arises without requiring an asymmetric carbon center or stereocenter. Where the rotational barrier about the single bond is high enough, and interconversion between conformations is slow enough, separation and isolation of the isomeric species may be permitted. Atropisomers are enantiomers without a single asymmetric atom. The atropisomers are considered stable if the barrier to interconversion is high enough to permit the atropisomers to undergo little or no interconversion at room temperature for at least a week, preferably at least a year. In some embodiments, an atropisomeric compound of the invention does not undergo more than about 5% interconversion to its opposite atropisomer at room temperature during one week when the atropisomeric compound is in substantially pure form, which is generally a solid state. In some embodiments, an atropisomeric compound of the invention does not undergo more than about 5% interconversion to its opposite atropisomer at room temperature (approximately 25° C.) during one year. Preferably, the atropisomeric compounds of the invention are stable enough to undergo no more than about 5% interconversion in an aqueous pharmaceutical formulation held at 0° C. for at least one week.

The energy barrier to thermal racemization of atropisomers may be determined by the steric hindrance to free rotation of one or more bonds forming a chiral axis. Certain biaryl compounds exhibit atropisomerism where rotation around an interannular bond lacking C2 symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biphenyl compounds may exhibit this type of conformational, rotational isomerism. Such biphenyls are enantiomeric, chiral atropisomers where the sp2-sp2 carbon-carbon, interannular bond between the phenyl rings has a sufficiently high energy barrier to prevent free rotation, and where substituents W≠Y and U≠V render the molecule asymmetric.

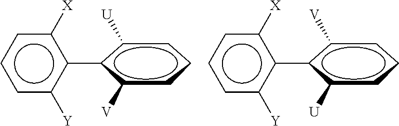

The steric interaction between X:U, X:V, and/or Y:V, Y:U is large enough to make the planar conformation an energy maximum. Two non-planar, axially chiral enantiomers then exist as atropisomers when their interconversion is slow enough such that they can be isolated free of each other. By one definition, atropisomerism is defined to exist where the isomers have a half-life, t_{1/2}, of at least 1000 seconds, which is a free energy barrier of 22.3 kcal mol⁻¹ (93.3 kJ mol⁻¹) at 300 K (Oki, M., “Recent Advances in Atropisomerism,” Topics in Stereochemistry (1983) 14:1). Bold lines and dashed lines in the figures shown above indicate those moieties, or portions of the molecule, which are sterically restricted due to a rotational energy barrier. Bolded moieties exist orthogonally above the plane of the page, and dashed moieties exist orthogonally below the plane of the page. The ‘flat’ part of the molecule (the left ring in each of the two depicted biphenyls) is in the plane of the page.

Compounds with axial chirality, such as chiral biphenyl rings, can be described using configurational nomenclature. For example, 2,2′; 6,6′-tetrasubstituted biphenyls are assigned the configurational descriptors as other axially chiral molecules. The molecules can be viewed from either end of the chiral axis and it leads to the same configurational descriptor (R or S). When, for instance, the compound of formula 3 is viewed from the left hand side along the 1-1′ bond, one arrives at projection 3.1 while the projection 3.2 is reached when the same molecule is now viewed from the right hand end along the 1′-1 bond. These projections conform to (S) configuration.

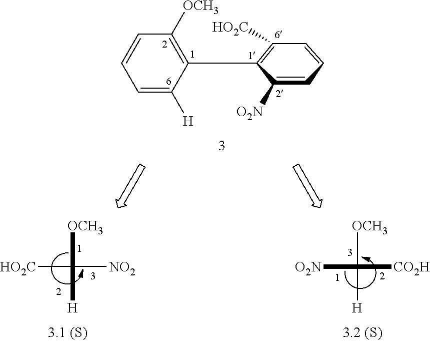

The S designation is assigned by applying sequence rules to name compounds with axial chirality. These rules are applied to primarily the ortho substituents of the biphenyl ring. The two linked rings may be represented by a horizontal and a vertical line. The lines represent the two orthogonal rings; and the ends of the lines represent the substituents at the four ortho positions of the two linked rings. These lines thus join each pair of ortho substituents. The two groups on the nearest ring (the ‘front’ line) take precedence over the two far groups. Within the pair, substituents are assigned priorities using the same priority rules used for describing R and S enantiomers of a chiral center. For example, in the projection formula 3.1 above, the perspective is viewing the molecule from the left side, looking down the axis from 1 to 1′. The near ring is represented by the bold vertical line connecting —OCH₃ and H, which are numbered 1 and 2, respectively, since —OCH₃ has a higher priority over H. The horizontal line represents the ring containing NO₂ and CO₂H, which are numbered 3 and 4, respectively, based on their priority. Thus, the sequence 1->2->3, reveals the configurational descriptor, which in this example is S, because following the numerical sequence in order requires going counter clockwise around the center of the diagram. As done for enantiomers, the numbered substituents are then taken in sequence by traveling either clockwise or counterclockwise around the point where the two lines intersect. If the path around the center point had been clockwise, the atropisomer would be designated R, just as it is for enantiomers of a stereocenter.

The same S configuration is deduced from viewing the molecule from the opposite end of the 1-1′ axis, as shown in FIG. 3.2. From this perspective, the ring containing the ortho NO₂ and ortho CO₂H is closer to the viewer and is represented by the bold horizontal line. The ring containing ortho OCH₃ and ortho H is further from the viewer and is represented by the vertical line.

In this biphenyl example, only the four ortho substituents are selected for nomenclature purposes. In the case wherein two ortho substituents in a ring are identical, the priority is given by considering meta substituents in the same ring.

This type of nomenclature assignment will be applied to the atropisomers described herein. For instance, formula 4, which is representative of a portion of the some of the compounds herein, such as formula 2(S), is assigned an absolute configuration of S as shown below.

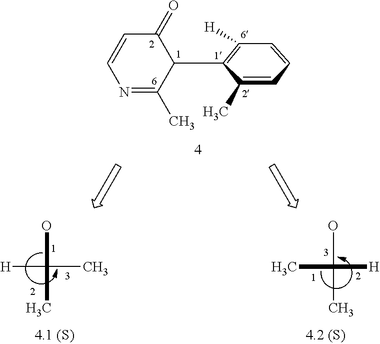

For purposes of the invention, the atropisomers are preferably sufficiently stable to be stored and used without substantial thermal interconversion. Typically, the atropisomers have a half-life of greater than 1 week when in solid form at room temperature.

The atropisomeric compounds referred to herein are obtained and used as a single atropisomer, or as a mixture wherein one atropisomer is present in excess over the other to an extent of at least 50% enrichment. As used herein, an atropisomer “substantially free” of its corresponding enantiomer means that the composition contains at least 90% by weight of one atropisomer, and 10% by weight or less of the stereoisomeric atropisomer. In some embodiments, the composition contains at least 95% by weight of one atropisomer and 5% by weight or less of the stereoisomer. In some embodiments, the composition contains at least 98% by weight of one atropisomer and 2% by weight or less of the stereoisomer. In some embodiments, the composition contains at least 99% by weight of one atropisomer and 1% by weight or less of the stereoisomer. In some embodiments, the composition contains at least 99.5% by weight of one atropisomer and 0.5% by weight or less of the stereoisomer. In some embodiments, the compounds of the invention consist of or consist essentially of a single atropisomer.

The atropisomeric compounds of the invention are typically solid materials, and are optionally purified to greater than about 90% purity, even if they exist as a mixture of atropisomers. In certain embodiments, the atropisomeric compound of the invention is substantially free of proteinaceous materials, or any materials having a molecular weight over about 1000 amu. Typically, they are at least 90% pure (chemically pure, regardless of optical purity), and preferably at least 95% chemically pure.

In some embodiments, the compositions and methods of the invention utilize an optically active form of the compounds described, meaning in each instance, the compound is optically active and contains predominantly the S-stereoisomer, such as 2(S), although it may contain the R-stereoisomer, such as 2(R), as a minor component. In other embodiments, the compound is optically active and contains predominantly the R-stereoisomer, such as 2(R), although it may contain the S-stereoisomer, such as 2(S), as a minor component.

For clarity, where a dosage of a compound is described herein, the dosage refers to the weight of the compound including each stereoisomer that is present. Thus, a dosage of 100 mg of formula 2(S) as used herein, for example, refers to the weight of the mixture of stereoisomers rather than the weight of the S-stereoisomer specifically. It could, for example, refer to 100 mg of a 9:1 mixture of S and R stereoisomers, which would contain about 90 mg of the S stereoisomer, or to 100 mg of a 19:1 mixture of S and R stereoisomers, which would contain about 95 mg of the S stereoisomer.

In certain embodiments, the compound is preferably used as a non-racemic mixture wherein the S isomer is the major component of the mixture. Typically such mixture will contain no more than about 10% of the R isomer, meaning the ratio of S to R isomers is at least about 9:1, and preferably less than 5% of the R-isomer, meaning the ratio of S to R enantiomers is at least about 19:1. In some embodiments the compound used has less than 2% R enantiomer, meaning it has an enantiomeric excess of at least about 96%. In some embodiments, the compound has an enantiomeric excess of at least 98%. In some embodiments, the compound has an enantiomeric excess of at least 99%.

In certain embodiments, the compound is preferably used as a non-racemic mixture wherein the R isomer is the major component of the mixture. Typically such mixture will contain no more than about 10% of the S isomer, meaning the ratio of R to S isomers is at least about 9:1, and preferably less than 5% of the S-isomer, meaning the ratio of R to S enantiomers is at least about 19:1. In some embodiments the compound used has less than 2% S enantiomer, meaning it has an enantiomeric excess of at least about 96%. In some embodiments, the compound has an enantiomeric excess of at least 98%. In some embodiments, the compound has an enantiomeric excess of at least 99%.

An atropisomer which is present “in excess” of its corresponding enantiomer or an “enantioenriched mixture” means that the atropisomer is present in an amount greater than its enantiomer, making the atropisomer mixture optically active. Typically this means the compound present “in excess” predominates by at least a 60/40 ratio over its enantiomer. It includes having a single atropisomer with none of its opposite atropisomer.

The compound of formula 2 has two atropisomers represented by formulas 2(S) and 2(R). In one embodiment, formula 2 where specific stereochemistry is not indicated represents a mixture of equal amounts of the two atropisomers 2(S) and 2(R). Formula 2(R) and formula 2(S) represent the individual stereoisomers, where 2(R) is the enantiomer of formula 2(S) and vice versa.

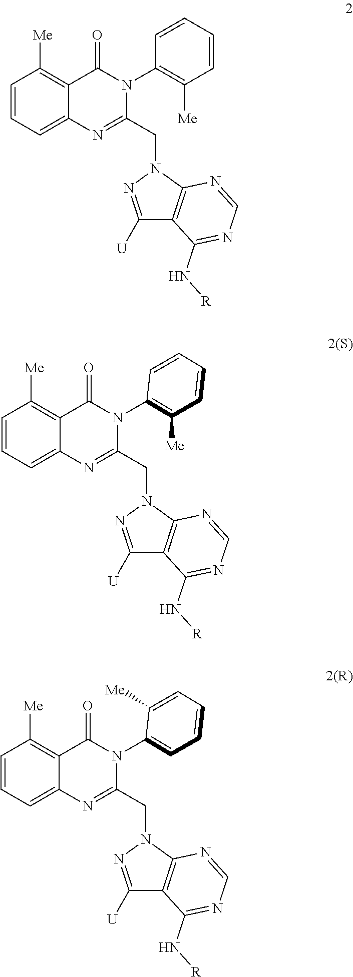

The invention relates to selective PI3Kδ inhibitors and methods to treat inflammatory conditions with compounds that are selective PI3Kδ inhibitors. In particular, compounds of the invention exist as separable atropisomers and the invention provides separated atropisomers having unexpected advantages over mixtures of atropisomers for use in treatment of inflammation. The compounds, compositions, and methods of the invention are therapeutically beneficial in treating inflammatory conditions.

The compounds of the invention are optically active versions of formula 1 or of formula 5 as described herein:

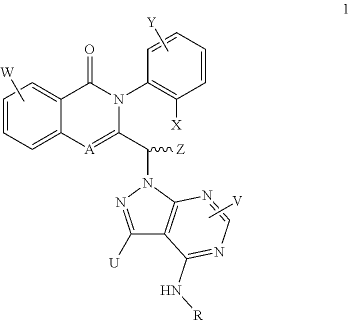

wherein W, Y, and V are each independently H, halo, R′, OR¹, CF₃, CN, where each R¹ is independently H or C1-C4 alkyl;

A is CH or N;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof.

In specific embodiments of the invention, W is H, F, Cl, Me, or OMe, and can be at any position on the phenyl ring where it is attached. In some embodiments it is Me or Cl or F, and is located at position 5 or 6 of the quinazolinone system using conventional numbering where the two nitrogen atoms are positions 1 and 3, and the carbonyl is position 4.

In specific embodiments of the foregoing compounds, Y is H or F. Y can be at any position on the ring that is available for substitution, but is not at the ‘ortho’ position adjacent to the point of attachment of the phenyl ring with the quinazolinone N when Y is the same as X. In preferred embodiments, Y is H.

In some embodiments of the foregoing compounds, A is CH; in other embodiments A is N.

In some embodiments of the foregoing compounds, X is Cl, Me or OMe. In some embodiments, X is preferably Me.

In some embodiments of the foregoing compounds, V is H or Me, and in preferred embodiments V is H.

In some embodiments of the foregoing compounds, R is H. In other embodiments, R is C1-C4 acyl.

In some embodiments, Z is H. Where Z is other than H, the linker between the pyrazole ring and the quinazolinone ring provides a second chiral center, and the compound of formula 1 exists as a mixture of diastereomers. Where that linker is chiral, the compound can be used as a mixture of the R and S isomers of the linker, or it can be separated. Where the linker is chiral and the isomers are separated, it is preferable to use the (S) isomer. In those embodiments, Z is frequently Me or Et.

In the foregoing compounds, U can be aryl, alkenyl or alkynyl, and can be substituted. In certain embodiments, U is alkynyl of the formula —C≡C-G, where G is H, aryl or C1-C4 alkyl, and aryl and alkyl are optionally substituted. In some embodiments, G is C1-C4 alkyl, which is optionally substituted with up to three substituents selected from the group consisting of halo, CN, OR′, SR′, NR′₂, CONR′₂, and COOR′, where each R′ is independently H or C1-C4 alkyl, and optionally one of the up to three substituents can be an optionally substituted aryl group, selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl. and optionally substituted with up to three substituents selected from the group consisting of halo, CN, OR′, SR′, NR′₂, CONR′₂, and COOR′, where each R′ is independently H or C1-C4 alkyl.

In other embodiments, G is a 5-10 atom monocyclic or bicyclic aromatic group containing up to three heteroatoms selected from N, O and S as ring members and optionally substituted. In some embodiments, G is selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl. In some embodiments when G is aryl, it is optionally substituted with up to three substituents selected from the group consisting of halo, CN, OR′, SR′, NR′₂, CONR′₂, and COOR′, where each R′ is independently H or C1-C4 alkyl. Preferred substituents for A when it is an aryl system include —OH, halo (especially F or Cl), and Me. In some embodiments where G is aryl, it is phenyl.

In certain embodiments of the compounds of formula 1,
W is H, F, Cl, Me, or OMe;
Y is H or F;
X is Cl, Me or OMe;
V is H or Me; and
U is optionally substituted alkynyl or aryl.

In one aspect, the invention provides an optically active compound comprising an atropisomer of formula 1(S)

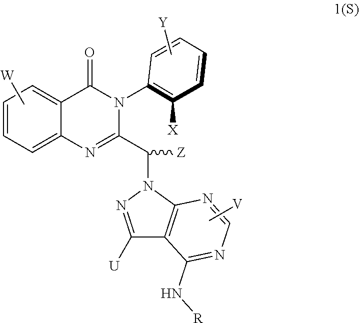

wherein A, U, V, W, X, Y, Z and R are as described above for formula 1; or a pharmaceutically acceptable salt thereof; and wherein the atropisomer of formula 1(S) is present in excess of its corresponding enantiomer of formula 1(R)

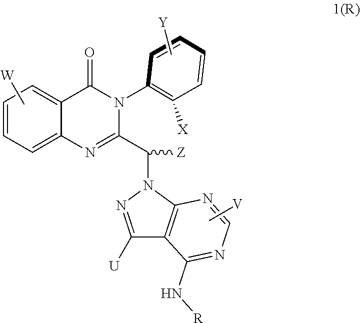

where A, U, V, W, X, Y, Z and R are as described above for formula 1.

In one embodiment, the atropisomer of formula 1(S) is substantially free of its corresponding atropisomer of formula 1(R).

In another aspect, the invention provides an optically active compound comprising an atropisomer of formula 1(R)

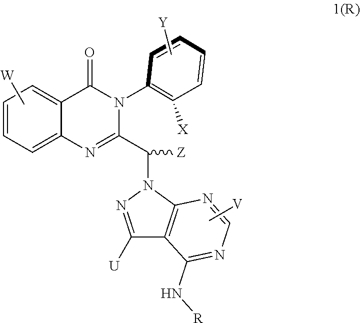

wherein A, U, V, W, X, Y, Z and R are as described above for formula 1; or a pharmaceutically acceptable salt thereof; and wherein the atropisomer of formula 1(R) is present in excess of its corresponding enantiomer of formula 1(S)

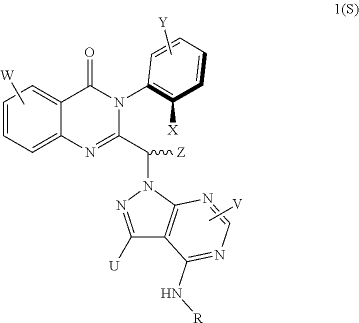

wherein A, U, V, W, X, Y, Z and R are as described above for formula 1.

In one embodiment, the atropisomer of formula 1(R) is substantially free of its corresponding atropisomer of formula 1(S).

In yet another aspect, the invention provides an optically active compound comprising an atropisomer of formula 2(S)

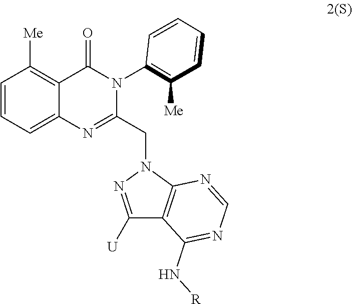

or a pharmaceutically acceptable salt thereof; and wherein the atropisomer of formula 2(S) is present in excess of its corresponding enantiomer of formula 2(R)

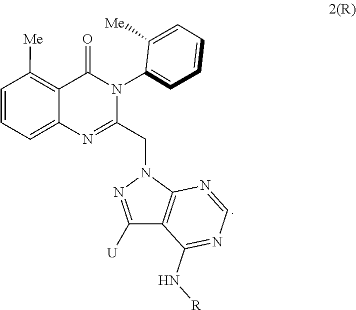

In compounds of formula 2, 2(R), and 2(S), U can be the same as for formula 1 above. In some embodiments of compounds within formula 2, including 2(R) and 2(S), U is an optionally substituted alkynyl or aryl group. In certain of these embodiments, U is alkynyl of the formula —C≡C-G, where G is H, aryl or C1-C4 alkyl, and aryl and alkyl are optionally substituted. In some embodiments, G is C1-C4 alkyl, which is optionally substituted with up to three substituents selected from the group consisting of halo, CN, OR′, SR′, NR′₂, CONR′₂, and COOR′, where each R′ is independently H or C1-C4 alkyl, and optionally one of the up to three substituents can be an optionally substituted aryl group, selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl, each of which is optionally substituted with up to three substituents selected from the group consisting of halo, CN, OR′, SR′, NR′₂, CONR′₂, and COOR′, where each R′ is independently H or C1-C4 alkyl.

In other embodiments of compounds within formula 2, G is a 5-10 atom monocyclic or bicyclic aromatic group containing up to three heteroatoms selected from N, O and S as ring members and optionally substituted. In some embodiments, G is selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl. In some embodiments when G is aryl, it is optionally substituted with up to three substituents selected from the group consisting of halo, CN, OR′, SR′, NR′₂, CONR′₂, and COOR′, where each R′ is independently H or C1-C4 alkyl. Preferred substituents for G when it is an aryl system include —OH, halo (especially F or Cl), and Me. In some embodiments, when G is aryl it is phenyl.

In compounds within formula 2, R can be H or C1-C4 acyl, as for formula 1; in preferred embodiments, R is H.

In one embodiment, the atropisomer of formula 2(S) is substantially free of its corresponding atropisomer of formula 2(R).

In another aspect, the invention provides an optically active compound comprising an atropisomer of formula 2(R)

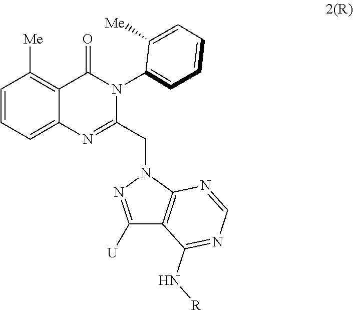

or a pharmaceutically acceptable salt thereof; and wherein the atropisomer of formula 2(R) is present in excess of its corresponding enantiomer of formula 2(S)

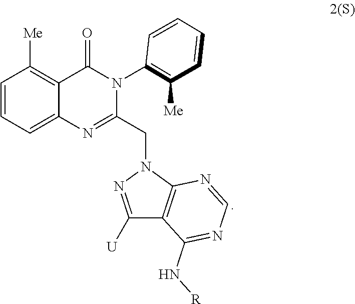

In one embodiment, the atropisomer of formula 2(R) is substantially free of its corresponding atropisomer of formula 2(S).

In specific embodiments of compounds of formula 2(R) or 2(S), R in formula 2 is H, and U is selected from the following groups:

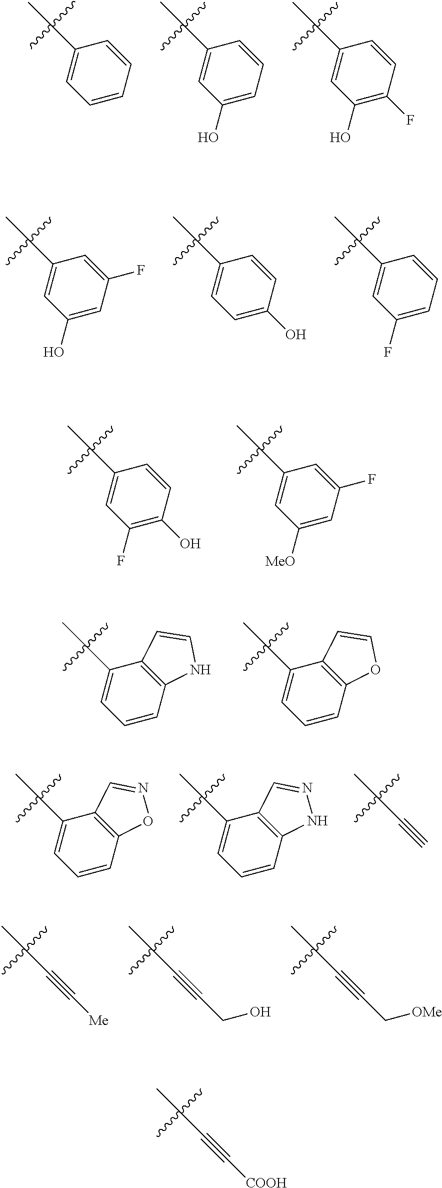

In another aspect, the invention provides compounds of formula 5 as well as pharmaceutically acceptable salts of these compounds:

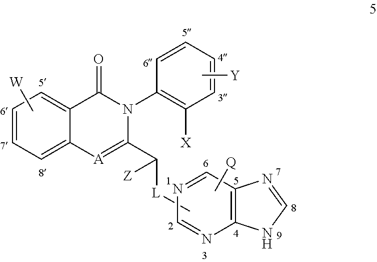

wherein W is an optional substituent that can be halo, C1-C4 alkyl, C1-C4 alkoxy, or CF₃;

L can be NR² or S or a bond, and can be attached to position 6 or 9 of the purine ring;

Z can be H or C1-C4 alkyl, or if L is NR², Z and N can be linked together to form a 5-6 membered optionally substituted ring;

A is CH or N;

Q can be H, Me, OMe, halo, or NFL or U, and is attached to the purine at position 2, 6, or 8, or at position 2 or 8 if L is attached at position 6;
U can be aryl, heteroaryl, cycloalkyl, heterocycloalkyl, alkyl, alkenyl, or alkynyl, each of which is optionally substituted;

X is Me, CF₃, Cl, CN, or Br;

Y can be H, C1-C4 alkyl, halo, CF₃, OMe, OH, NH₂, NHAc, or CN;

with the proviso that W and X are not both Me when W is at position 5′ and L is a bond;

or a pharmaceutically acceptable salt thereof.

In these compounds, X is large enough to induce atropisomers formed by hindered rotation about the N-phenyl(X) bond that are stable and separable, providing a center of chirality. If Z is not H, the compounds can also contain a chiral center where Z is attached; optionally, the compounds can be a single isomer at this chiral center, e.g., at least about 90% and preferably 95% or more one enantiomer at this chiral center. In preferred embodiments, the chiral center bearing Z is predominantly in the S configuration. The selection of a single atropisomer in combination with the selection of the S enantiomer at the chiral center bearing Z obviates complex separation and pharmacokinetic variations that would arise from use of such compounds as mixtures of diastereomers, while preserving the most active isomer.

In these compounds, Z can be H, especially when L is a bond. When L is NH, Z is frequently Me or Et, introducing a chiral center; and the chiral center is preferably in the S configuration.

Where L is a bond, it represents a single bond between the carbon to which Z is attached and an atom of the purine ring system. In some embodiments, it connects to position 6 of the purine ring system; in other embodiments, it connects to position 9 of the purine ring system. When L is attached at position 6, Q if other than H is often at position 3 or 8, preferably position 3. When L is attached at position 9 of the purine, Z if other than H is often at position 6 or at position 8, preferably at position 6.

W is optional, so it can be absent. When present, it can be at position 5′, 6′, 7′, or 8; preferably it is at position 5′ or 6′, and frequently at position 5′. Often, W is Me or Cl or F, or W is absent. X is preferably Me.

Some preferred embodiments of these compounds include compounds of formula 5a and 5b, wherein A, Q, W, X, Y, Z and L are as defined for formula 5:

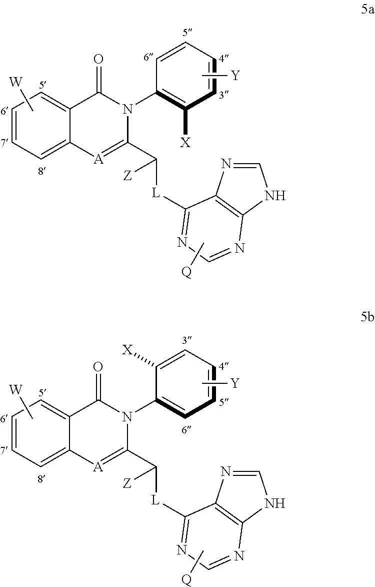

and compounds of formula 6a and 6b, where A, Q, W, X, Y, Z and L are as defined for formula 5:

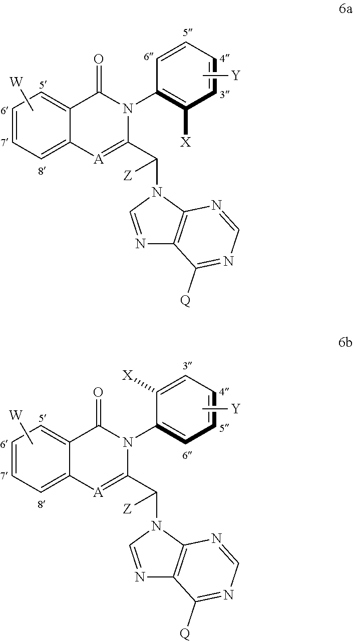

In these compounds, X is often Me; W is often Me, F or Cl and is preferably at position 5′ or 6′, or W may be absent; Y may be H; and Z can be H, Me, or Et. Frequently Q is H in formula 5a or 5b, and H or NH₂ in formula 6a or 6b. A can be CH or it can be N in these compounds.

In some embodiments of these compounds, Q is H or NH₂. In other embodiments, Q is U. Typically, U is an optionally substituted alkynyl or aryl group. In certain of these embodiments, U is alkynyl of the formula —C≡C-G, where G is H, aryl or C1-C4 alkyl, and aryl and alkyl are optionally substituted. In some embodiments, G is C1-C4 alkyl, which is optionally substituted with up to three substituents selected from the group consisting of halo, CN, OR′, SR′, NR′₂, CONR′₂, and COOR′, where each R′ is independently H or C1-C4 alkyl, and optionally one of the up to three substituents can be an optionally substituted aryl group, selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl, each of which is optionally substituted with up to three substituents selected from the group consisting of halo, CN, OR′, SR′, NR′₂, CONR′₂, and COOR′, where each R′ is independently H or C1-C4 alkyl.

In other embodiments of compounds where Q is U, G is a 5-10 atom monocyclic or bicyclic aromatic group containing up to three heteroatoms selected from N, O and S as ring members and optionally substituted. In some embodiments, G is selected from phenyl, pyridinyl, pyrimidinyl, thienyl, furanyl, imidazolyl, pyrazolyl, oxazolyl, isoxazolyl, thiazolyl, isothiazolyl, naphthyl, benzofuranyl, benzothienyl, benzimidazolyl, benzopyrazolyl, and indolyl. In some embodiments when G is aryl, it is optionally substituted with up to three substituents selected from the group consisting of halo, CN, OR′, SR′, NR′₂, CONR′₂, and COOR′, where each R′ is independently H or C1-C4 alkyl. Preferred substituents for G when it is an aryl system include —OH, halo (especially F or Cl), and Me. In some embodiments, when G is aryl it is phenyl.

In compounds of formula 6a and 6b, Z is preferably H, and Q is preferably NH₂. X is frequently Me or CF₃. A may be CH or it may be N; and W is typically Me, F or Cl, located at position 5′ or 6′, or W is absent.

In compounds of formula 5a and 5b, Z is preferably Me or Et, and Q is preferably H. X is frequently Me or CF₃. A may be CH or it may be N; and W is typically H, Me, F or Cl, located at position 5′ or 6′, or W is absent.

In another aspect, the invention provides a pharmaceutical composition comprising any of the optically active compounds described herein, and at least one pharmaceutically acceptable excipient. In particular embodiments, the optically active compound is 1(S) or 1(R). In other embodiments, the optically active compound is 2(S). In yet other embodiments, the optically active compound is 2(R). In still other embodiments, the compound is a compound of formula 5a, 5b, 6a or 6b as described herein.

As used herein, the term “alkyl” is defined as straight chained or branched hydrocarbon groups or cyclic hydrocarbon groups containing the indicated number of carbon atoms, typically methyl, ethyl, and straight chain and branched propyl and butyl groups, and cyclopropyl, cyclopentyl and cyclohexyl groups, as well as combination of straight chain, branched chain and cyclic groups, e.g., cyclopropylmethyl and norbornyl. The hydrocarbon group can contain up to 16 carbon atoms, preferably one to eight carbon atoms. The term “alkyl” includes cyclic, bicyclic, and “bridged alkyl,” i.e., a C6-C16 bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C3-C8 hydrocarbon group, e.g., cyclopropyl, cyclobutyl, cyclohexyl, and cyclopentyl.

The term “alkenyl” is defined identically as “alkyl,” except the hydrocarbon groups contain at least two carbons and at least one carbon-carbon double bond. The term “alkynyl” defined identically as “alkyl,” except the hydrocarbon groups contain at least two carbons and at least one carbon-carbon triple bond. “Cycloalkenyl” is defined similarly to cycloalkyl, except at least one carbon-carbon double bond is present in the ring.

The term “perfluoroalkyl” is defined as an alkyl group wherein each hydrogen atom is replaced by fluorine.

The term “alkylene” is defined as an alkyl group having a substituent, for example, the term “C1-3alkylenearyl” refers to an alkyl group containing one to three carbon atoms, and substituted with an aryl group. Similarly, “alkylene” when used without description of another group can refer to a divalent alkyl group, which can link two other structural features together, for example, CH₂ and (CH₂)₃ are 1-carbon and 3-carbon alkylene groups.

The term “halo” or “halogen” is defined herein to include fluorine, bromine, chlorine, and iodine. Often, fluoro or chloro is preferred.

The term “haloalkyl” is defined herein as an alkyl group substituted with one or more halo substituents, i.e., fluoro, chloro, bromo, iodo, or combinations thereof. Similarly, “halocycloalkyl” is defined as a cycloalkyl group having one or more halo substituents.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, halo alkyl, nitro, amino, alkylamino, acylamino, alkylthio, alkylsulfinyl, and alkylsulfonyl. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like.

The term “heteroaryl” is defined herein as a monocyclic or bicyclic ring system containing one or two aromatic rings and containing at least one nitrogen, oxygen, or sulfur atom in an aromatic ring and up to three such heteroatoms per ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include thienyl, furyl, pyridyl, oxazolyl, quinolyl, isoquinolyl, indolyl, triazolyl, isothiazolyl, isoxazolyl, imidazolyl, benzothiazolyl, pyrazinyl, pyrimidinyl, thiazolyl, and thiadiazolyl.

The term “C3-8heterocycloalkyl” is defined as monocyclic ring system containing one or more heteroatoms selected from the group consisting of oxygen, nitrogen, and sulfur. A “C3-8heterocycloalkyl” group also can contain an oxo group (═O) attached to the ring. Nonlimiting examples of “C3-8heterocycloalkyl” groups include 1,3-dioxolane, 2-pyrazoline, pyrazolidine, pyrrolidine, piperazine, a pyrroline, 2H-pyran, 4H-pyran, morpholine, thiomorpholine, piperidine, 1,4-dithiane, and 1,4-dioxane.

The term “hydroxy” is defined as —OH.

The term “alkoxy” is defined as —OR, wherein R is C1-C8 alkyl, C2-C8 alkenyl or C2-C8 alkynyl; each alkyl, alkenyl and alkynyl group is optionally substituted.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “hydroxyalkyl” is defined as a hydroxy group appended to an alkyl group.

The term “alkylthio” is defined as —SR, wherein R is alkyl.

The term “alkylsulfinyl” is defined as R—SO, wherein R is alkyl.

The term “alkylsulfonyl” is defined as R—SO₂, wherein R is alkyl.

The term “amino” is defined as —NH₂, and the term “alkylamino” is defined as —NR₂, wherein at least one R is alkyl, alkenyl or alkynyl, and the second R is alkyl, alkenyl, alkynyl or hydrogen.

The term “acylamino” is defined as RC(═O)N, wherein R is alkyl, alkenyl, alkynyl or aryl, heteroaryl, or heterocyclyl.

The term “nitro” is defined as —NO₂.

The term “trifluoromethyl” is defined as —CF₃.

The term “trifluoromethoxy” is defined as —OCF₃.

The term “cyano” is defined as —CN.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where two R′ are included in one such substituent, as in —NR′₂ by way of example only, the two R′ can be linked together to form a 4-7 membered ring, optionally containing an additional N, O or S atom as a ring member and optionally substituted as allowed for the groups linked together to form the ring.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

“Heteroforms” as used herein refers to a modified alkyl, alkenyl, aryl, etc., wherein at least one heteroatom selected from N, O and S replaces at least one carbon atom in the hydrocarbon group being described. Typically a heteroform has only one such heteroatom replacing one carbon atom.

In one embodiment, the composition of the invention comprising a therapeutically effective amount of the optically active compound comprising an optically active atropisomer described herein for the treatment of a condition, wherein the condition is characterized by inflammation. In some embodiments, the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

In another aspect, the invention provides a method of treating a condition in a mammal, wherein the condition is characterized by inflammation. In some embodiments, the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (MD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury; which comprises administering to said mammal a therapeutically effective amount of an optically active atropisomer described herein. In certain embodiments, the optically active compound is represented by formula 2(S). In other embodiments, the optically active compound is represented by formula 2(R). In some embodiments, the mammal is one identified as in need of treatment for the disorder. In some embodiments, the mammal is one at risk of the condition and the compound or composition is administered to reduce or prevent the occurrence of inflammation.

A method of the present invention can be employed to treat subjects therapeutically or prophylactically who have or can be subject to an inflammatory condition. Examples of inflammatory conditions include but are not limited to arthritic diseases such as rheumatoid arthritis (RA), osteoarthritis (OA), gouty arthritis, spondylitis, and reactive arthritis; Behçet's syndrome; sepsis; septic shock; endotoxic shock; gram negative sepsis; gram positive sepsis; toxic shock syndrome; multiple organ injury syndrome secondary to septicemia, trauma, or hemorrhage; ophthalmic disorders including but not limited to allergic conjunctivitis, vernal conjunctivitis, uveitis, and thyroid-associated ophthalmopathy; eosinophilic granuloma; pulmonary or respiratory conditions including but not limited to asthma, chronic bronchitis, allergic rhinitis, adult respiratory distress syndrome (ARDS), severe acute respiratory syndrome (SARS), chronic pulmonary inflammatory diseases (e.g., chronic obstructive pulmonary disease), silicosis, pulmonary sarcoidosis, pleurisy, alveolitis, vasculitis, pneumonia, bronchiectasis, hereditary emphysema, and pulmonary oxygen toxicity; ischemic-reperfusion injury, e.g., of the myocardium, brain, or extremities; fibrosis including but not limited to cystic fibrosis; keloid formation or scar tissue formation; atherosclerosis; autoimmune diseases including but not limited to systemic lupus erythematosus (SLE), lupus nephritis, autoimmune thyroiditis, multiple sclerosis, some forms of diabetes, and Reynaud's syndrome; tissue or organ transplant rejection disorders including but not limited to graft versus host disease (GVHD) and allograft rejection; chronic or acute glomerulonephritis; inflammatory bowel diseases including but not limited to Crohn's disease, ulcerative colitis and necrotizing enterocolitis; inflammatory dermatitis including but not limited to contact dermatitis, atopic dermatitis, psoriasis, and urticaria; fever and myalgias due to infection; central or peripheral nervous system inflammatory conditions including but not limited to meningitis (e.g., acute purulent meningitis), encephalitis, and brain or spinal cord injury due to minor trauma; Sjögren's syndrome; diseases involving leukocyte diapedesis; alcoholic hepatitis; bacterial pneumonia; community acquired pneumonia (CAP); Pneumocystis carinii pneumonia (PCP); antigen-antibody complex mediated diseases; hypovolemic shock; Type 1 diabetes mellitus; acute and delayed hypersensitivity; disease states due to leukocyte dyscrasia and metastasis; thermal injury; granulocyte transfusion associated syndromes; cytokine-induced toxicity; stroke; pancreatitis; myocardial infarction, respiratory syncytial virus (RSV) infection; and spinal cord injury.

In some embodiments, the inflammatory condition is selected from the group consisting of allergic rhinitis, asthma, atopic dermatitis, chronic obstructive pulmonary disease (COPD), multiple sclerosis (MS), rheumatoid arthritis (RA), and type 1 diabetes.

In another embodiment, the application disclose a method to treat cancer. In some embodiments, the cancer is a hematological malignancy and/or solid tumor. In another particular embodiment, the hematological malignancy is leukemia or lymphoma.

In some embodiments, lymphoma is a mature (peripheral) B-cell neoplasm. In specific embodiments, the mature B-cell neoplasm is selected from the group consisting of B-cell chronic lymphocytic leukemia/small lymphocytic lymphoma; B-cell prolymphocytic leukemia; Lymphoplasmacytic lymphoma; Marginal zone lymphoma, such as Splenic marginal zone B-cell lymphoma (+/− villous lymphocytes), Nodal marginal zone lymphoma (+/− monocytoid B-cells), and Extranodal marginal zone B-cell lymphoma of mucosa-associated lymphoid tissue (MALT) type; Hairy cell leukemia; Plasma cell myeloma/plasmacytoma; Follicular lymphoma, follicle center; Mantle cell lymphoma; Diffuse large cell B-cell lymphoma (including Mediastinal large B-cell lymphoma, Intravascular large B-cell lymphoma, and Primary effusion lymphoma); and Burkitt's lymphoma/Burkitt's cell leukemia.

In some embodiments, lymphoma is selected from the group consisting of multiple myeloma (MM) and non-Hodgkin's lymphoma (NHL), mantle cell lymphoma (MCL), follicular lymphoma, Waldenstrom's macroglobulinemia (WM) or B-cell lymphoma and diffuse large B-cell lymphoma (DLBCL).

In a further particular embodiment, leukemia is selected from the group consisting of acute lymphocytic leukemia (ALL), acute myeloid leukemia (AML), chronic lymphocytic leukemia (CLL), and small lymphocytic lymphoma (SLL). Acute lymphocytic leukemia is also known as acute lymphoblastic leukemia and may be used interchangeably herein. Both terms describe a type of cancer that starts from the white blood cells, lymphocytes, in the bone marrow. In another embodiment, the cancer is T-cell acute lymphoblastic leukemia.

In some embodiments, Non-Hodgkin's Lymphoma (NHL) falls into one of two categories, aggressive NHL or indolent NHL. Aggressive NHL is fast growing and may lead to a patient's death relatively quickly. Untreated survival may be measured in months or even weeks. Examples of aggressive NHL includes B-cell neoplasms, diffuse large B-cell lymphoma, T/NK cell neoplasms, anaplastic large cell lymphoma, peripheral T-cell lymphomas, precursor B-lymphoblastic leukemia/lymphoma, precursor T-lymphoblastic leukemia/lymphoma, Burkitt's lymphoma, Adult T-cell lymphoma/leukemia (HTLV1+), primary CNS lymphoma, mantle cell lymphoma, polymorphic post-transplantation lymphoproliferative disorder (PTLD), AIDS related lymphoma, true histiocytic lymphoma, and blastic NK-cell lymphoma. The most common type of aggressive NHL is diffuse large cell lymphoma.

Indolent NHL is slow growing and does not display obvious symptoms for most patients until the disease has progressed to an advanced stage. Untreated survival of patients with indolent NHL may be measured in years. Non-limiting examples include follicular lymphoma, small lymphocytic lymphoma, marginal zone lymphoma (such as extranodal marginal zone lymphoma (also called mucosa associated lymphoid tissue—MALT lymphoma), nodal marginal zone B-cell lymphoma (monocytoid B-cell lymphoma), splenic marginal zone lymphoma), and lymphoplasmacytic lymphoma (Waldenstrom's macroglobulinemia).

In another aspect, the invention provides an optically active atropisomer obtained by chiral chromatographic separation of an enantiomeric mixture of formula 1

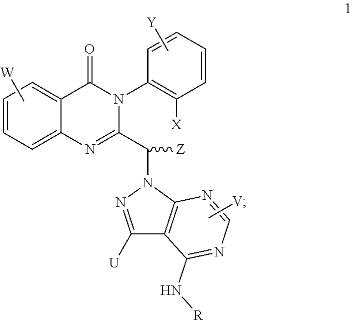

wherein U, V, W, X, Y, Z and R are as described above for formula 1; or a pharmaceutically acceptable salt thereof; wherein an enantiomeric mixture of formula 1 is purified using a normal phase chiral column, and two peaks, A and B, are resolved, wherein peak A and peak B represent the atropisomers, 1(S) and 1(R), respectively,

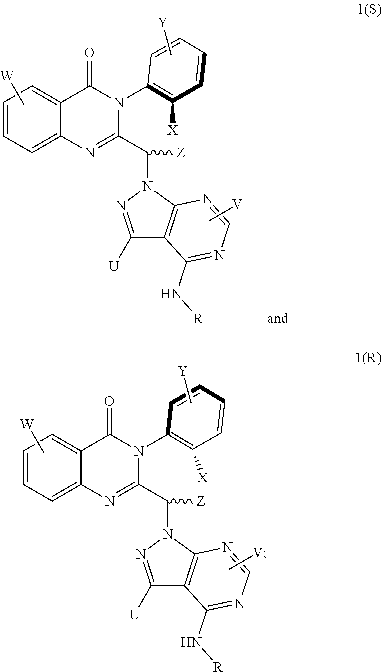

wherein the predominant optically active atropisomer obtained is the first compound to elute from the column. Preferably 1(S) or 1(R) is obtained substantially free of its enantiomer.

In one embodiment, the predominant optically active atropisomer obtained is the compound of formula 1(S) substantially free of the compound of formula 1(R). In another embodiment, the predominant optically active atropisomer obtained is the compound of formula 1(R) substantially free of the compound of formula 1(S).

In yet another aspect, the invention provides an optically active atropisomer obtained by chiral chromatographic separation of an enantiomeric mixture of formula 1

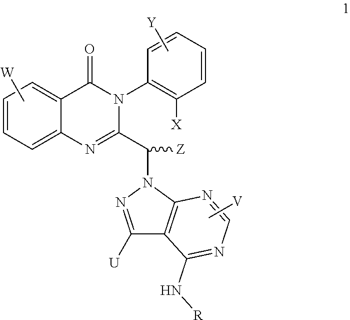

wherein U, V, W, X, Y, Z and R are as described above for formula 1; or a pharmaceutically acceptable salt thereof; wherein an enantiomeric mixture of formula 1 is purified using a normal phase chiral column, and two peaks, A and B, are resolved, wherein peak A and peak B represent the atropisomers, 1(S) and 1(R), respectively,

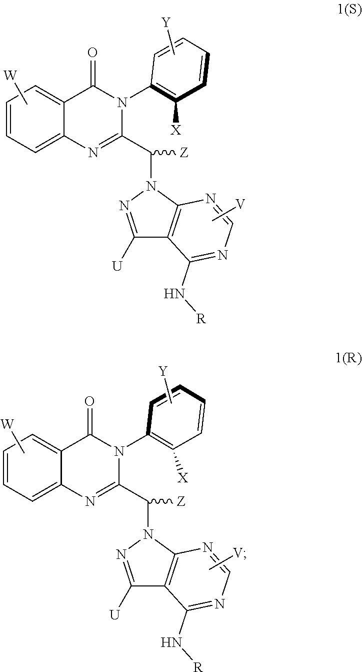

and

wherein the predominant optically active atropisomer obtained is the second compound to elute from the column. Preferably 1(S) or 1(R) is obtained substantially free of its enantiomer.

In one embodiment, the predominant optically active atropisomer obtained is the compound of formula 1(S) substantially free of the compound of formula 1(R). In another embodiment, the predominant optically active atropisomer obtained is the compound of formula 1(R) substantially free of the compound of formula 1(S).

In one embodiment, the compound of the invention is purified using a chiral chromatographic column. In certain embodiments, the chiral column has a normal phase. In another embodiment, the chiral column has a reverse phase.

The atropisomers of formula 1 or 2 can be separated by normal phase chiral HPLC methods resulting in two resolved peaks by methods know in the art, and the two peaks will provide samples of each of the enantiomers of 2 substantially free of the opposite enantiomer. The absolute configuration of each isolated compound can be elucidated from x-ray crystallographic data. The elution order of the peaks is typically reversed when a reverse phase column is used.

The in vitro activity of compounds 1 or 2 and their atropisomers, e.g., 2(S) and 2(R), can have similar profiles with respect to inhibition of various isoforms of p110 inhibition or they may have different profiles. Even though their in vitro potency may be similar, there can be surprising in vivo differences observed between 1(S) and 1(R), or 2(S) and 2(R), as discovered in pharmacokinetic studies. Selecting a specific atropisomer can optimize in vivo efficacy based on PK differences alone or in combination with in vitro efficacy for the inflammation uses of interest. Likewise, compounds 5a and 5b, or compounds 6a and 6b, can have similar activity at the PI3K target site(s) but still have quite different pharmacokinetic properties. Because there are significant advantages to using a single atropisomer, both for registration purposes and for therapeutic predictability and consistency, using a single atropisomer is often critical.

In order to perform the pharmacokinetic studies, formula 2 can be radiolabeled using ¹⁴C at the ortho-methyl group on the phenyl at position 3 of the quinazolinone ring. Radiolabeled 2 (where U is defined as for formula 1):

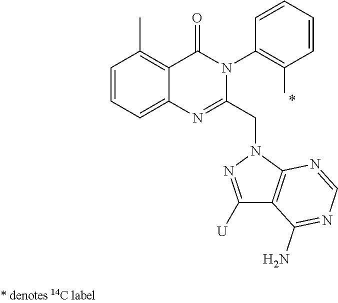

The tagged racemic mixture or separated atropisomers can be administered in rats, dogs, and human subjects through oral and i.v. routes. The compounds can be dissolved for administration, e.g. in PEG 100, so any difference in dissolution rates would not play a role in the pharmacokinetic profile of the compounds. After administration of the compound, blood plasma of the subjects can be sampled over time and evaluated by analytical HPLC methods developed to identify and measure concentrations of formula 2(S) or 2(R) present in the sample.

The in vivo differences between compounds 2(S) and 2(R) can be examined in human subjects. In some embodiments, the maximum concentration (Cₘₐₓ) of 2(S) is more than 2 times as great as the maximum concentration for 2(R), and the 2(S) isomer is preferred. Although the concentration of the compounds in the blood plasma decreases over the 72 hour period, the difference in concentration of the two compounds can be maintained, if not further broadened. This difference in compound concentration in the blood appears to broaden because formula 2(S) decreases more gradually over time whereas formula 2(R) appears to be removed from the blood relatively more quickly. At a dose of 10 mg, the maximum blood plasma concentration of formula 2(S) can be still about double the maximum concentration of formula 2(R).

Without being bound by theory, the lower exposure provided by 2(R) compared to 2(S) can include a difference due to absorption and elimination between the two compounds. Without being bound by theory, another explanation for the difference in exposure can be that 2(R) interconverted to 2(S) over time. The difference may also be related to more rapid metabolism of 2(R). Regardless of the reasons, 2(R) is less available in plasma (circulation) that 2(S) when administered orally.

Formula 2(S) can offer the advantages of a longer half-life in vivo, reduced dosing amount and increased exposure in vivo. However, the pharmacokinetic characteristics of 2(R) also provide certain advantages for its use in some situations and subjects. The different pharmacokinetic profile of 2(R) can provides a slower delivery of the 2(S) isomer if interconversion occurs, producing slow formation of 2(S). For example, the interconversion of 2(R) to 2(S), as discussed previously, may provide a way to deliver a delayed exposure to formula 2(S), with a shortened exposure to high plasma concentration of active drug due to the short half-life of 2(R). Thus, the slower onset profile of formula 2(R) may be advantageous when a drug that has a greater area under the curve (AUC) profile is desired rather than a drug with a large Cₘₐₓ value. Accordingly, in certain embodiments, compounds, methods and compositions of the invention comprise 2(S). In other embodiments, the compounds, compositions and methods of the invention comprise 2(R).

Chiral resolution of enantiomers can be carried out by methods of high pressure liquid chromatography (HPLC), crystallization of diastereomers or diastereomeric salts, or the use of enzymes, using conventional methods. Described herein are chiral resolution methods that employ HPLC to provide the compounds of the invention. For instance, mixtures of the atropisomers of formula 2 can be separated into compounds of the formulas 2(S) and 2(R). Because it is conveniently correlated with analytical methods, herein are described methods of chiral chromatographic separation to separate and isolate the individual atropisomers.

One of ordinary skill in the art will understand that many types of instruments, columns and eluents can be used to separate the individual atropisomers. Suitable HPLC instruments are configured according to methods well known to those of ordinary skill in the art. Such configuration invariably includes a pump, injection port and a detector.

Chromatographic columns may be characterized as ‘normal phase’ or ‘reverse phase’. In general, normal phase columns have a polar stationary phase and reverse phase columns have a non-polar stationary phase. Suitable chiral columns can be purchased prepackaged or can be packed by one of ordinary skill in the art. Suitable chiral columns include chiral CHIRALPAK®IA, IB, AD-H, AS, AD-RH, AS-RH and IC columns as well as CHIRALCEL®OD-H, OB-H, OF, OG, OJ-RH and OJ which can be purchased from Chiral Technologies Inc., 730 Springdale Drive, PO Box 564, Exton, Pa. 19341. The packing composition for CHIRALPAK® IA columns is amylose tris(3,5-dimethylphenylcarbamate) immobilized on 5 μM silica-gel. One of ordinary skill in the art will appreciate that many other chiral columns, purchased from other vendors, would be adequate to separate the isomers of the invention, and that in view of the invention described herein, such methods can be expected to provide separated isomers that maintain their chiral configuration. The packing material for such chiral columns can also be purchased in different bead sizes. Suitable bead sizes for preparative separations are typically about 20 microns in diameter or less. Suitable bead sizes for analytical separation are frequently about 10 microns in diameter or less.

One of ordinary skill in the art will understand that the appropriate mobile phase used in an HPLC method can be selected from various combinations and ratios of solvents. A suitable mobile phase is determined according to methods well known to those of ordinary skill in the art. The mobile phase may include organic solvents such as alkanes, alcohols, ethers, chlorinated solvents as water, and buffered water. Non-limiting examples of organic solvents include hexanes, n-hexane, methanol, ethanol, butanol, isobutanol, propanol, isopropanol (IPA), acetonitrile, N,N-dimethylformamide (DMF), tetrahydrofuran (THF), methyl-t-butyl ether, trichloromethane, dichlormethane, chloroform, 1,4-dioxane, toluene, acetone, methyl acetate and ethyl acetate. For basic or acidic samples, an additive may be incorporated into the mobile phase in order to optimize chiral separation. Primary amines, such as diethylamine (DEA), diisopropylamine, butyl amine, and triethylamine (TEA) may be used as bases. Non-limiting examples of acids include sulfuric acid, trifluoroacetic acid, hydrochloric acid, acetic acid, and formic acid. Other inorganic mobile phase additives may also be used, such as KPF6, NaClO₄, NaBF₄, or NaH₂PO₄. Non-limiting examples of mobile phase mixtures include 50:50:0.2 methanol/ethanol/DEA; 70:30:0.1 hexanes/ethanol/DEA; 70:30:0.1 hexanes/isopropanol/DEA; 40:60:0.06 hexanes/isopropanol/DEA; and 50:50, 60:40 or 70:30 water/acetonitrile. Non-limiting examples of mobile phases used for reverse phase screenings of basic compounds include 30:70 pH 9 borate/acetonitrile and 30:70 100 mM aqueous KPF₆/acetonitrile.

For a description of analytical or preparatory chromatographic methods, see Examples 2 and 3, respectively.

The relative efficacies of compounds as inhibitors of an enzyme activity (or other biological activity) can be established by determining the concentrations at which each compound inhibits the activity to a predefined extent, then comparing the results. Typically, the preferred determination is the concentration that inhibits 50% of the activity in a biochemical assay, i.e., the 50% inhibitory concentration or “IC50.” IC50 determinations can be accomplished using conventional techniques known in the art. In general, an IC50 can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC50 value. Analogously, other inhibitory concentrations can be defined through appropriate determinations of activity. For example, in some settings it can be desirable to establish a 90% inhibitory concentration, i.e., IC90.

“Treating” as used herein refers to preventing a disorder from occurring in an animal that can be predisposed to the disorder, but has not yet been diagnosed as having it; inhibiting the disorder, e.g., slowing or arresting its development; relieving the disorder, e.g., causing its regression or elimination; or ameliorating the disorder, i.e., reducing the severity of symptoms associated with the disorder. “Disorder” is intended to encompass medical disorders, diseases, conditions, syndromes, and the like, without limitation.

The methods of the invention embrace various modes of treating an animal subject, preferably a mammal, more preferably a primate, and still more preferably a human. Among the mammalian animals that can be treated are, for example, humans, companion animals (pets), including dogs and cats; farm animals, including cattle, horses, sheep, pigs, and goats; laboratory animals, including rats, mice, rabbits, guinea pigs, and nonhuman primates; and zoo specimens. Non-mammalian animals include, for example, birds, fish, reptiles, and amphibians. In general, any subject who would benefit from the compounds and compositions of the invention is appropriate for administration of the invention method.

Techniques for formulation and administration of pharmaceutical compositions can be found in Remington's Pharmaceutical Sciences, 18th Ed., Mack Publishing Co, Easton, Pa., 1990. The pharmaceutical compositions of the present invention can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions are formulated to contain suitable pharmaceutically acceptable carriers, and optionally can comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hanks' solution, Ringer's solution, or physiologically buffered saline. For tissue or cellular administration, penetrants appropriate to the particular bather to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT® series available from Röhm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethylene sorbitol and sorbitan esters, microcrystalline cellulose, aluminum metahydroxide, bentonite, agar-agar, gum tragacanth, and mixtures thereof.

Liposomes containing the active agent also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

Pharmaceutical compositions comprising the agent in dosages suitable for oral administration can be formulated using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or excipients, which include, without limitation:

a) diluents, such as sugars, including lactose, dextrose, sucrose, mannitol, or sorbitol;

b) binders, such as magnesium aluminum silicate, starch from corn, wheat, rice, potato, etc.;

c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;

d) disintegrating or solubilizing agents such as cross-linked polyvinyl pyrrolidone, starches, agar, alginic acid or a salt thereof, such as sodium alginate, or effervescent compositions;

e) lubricants, such as silica, talc, stearic acid or its magnesium or calcium salt, and polyethylene glycol;

f) flavorants and sweeteners;

g) colorants or pigments, e.g., to identify the product or to characterize the quantity (dosage) of active compound; and

h) other ingredients, such as preservatives, stabilizers, swelling agents, emulsifying agents, solution promoters, salts for regulating osmotic pressure, and buffers.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers. Dragee cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a pharmaceutically acceptable salt of a compound of the invention. Salts are often more soluble in aqueous or other protonic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metal (e.g., sodium or potassium) and alkaline earth (e.g., calcium or magnesium) cations.

Compounds of the invention that contain basic moieties can form pharmaceutically acceptable acid addition salts with suitable acids. For example, Berge, et al., J Pharm Sci (1977) 66:1, describe pharmaceutically acceptable salts in detail. The salts can be prepared in situ during the final isolation and purification of the compounds of the invention, or separately by reacting a free base function with a suitable acid.

Representative acid addition salts include, but are not limited to, acetate, adipate, alginate, citrate, aspartate, benzoate, benzenesulfonate, bisulfate, butyrate, camphorate, camphorolsulfonate, cinnamate, digluconate, formate, glycerophosphate, hemisulfate, heptanoate, hexanoate, fumarate, hippurate, hydroxyacetate, hydrochloride, hydrobromide, hydroiodide, 2-hydroxyethanesulfonate (isethionate), lactate, maleate, malonate, mandelate, methane sulfonate or sulfate, nicotinate, 2-naphthalenesulfonate, oxalate, pamoate, pectinate, persulfate, 3-phenylpropionate, picrate, pivalate, propionate, pyruvate, succinate, tartrate, thiocyanate, phosphate or hydrogen phosphate, glutamate, bicarbonate, salicylate, p-toluenesulfonate, and undecanoate.

Examples of inorganic acids include, but are not limited to, hydrochloric acid, hydrobromic acid, sulfuric acid, nitric acid, and phosphoric acid.

Basic addition salts can be prepared in situ during the final isolation and purification of the compounds of the invention or separately by reacting a carboxylic acid-containing moiety with a suitable base such as the hydroxide, carbonate, or bicarbonate of a pharmaceutically acceptable metal cation, or with ammonia or organic primary, secondary, or tertiary amine. Pharmaceutically acceptable basic addition salts include, but are not limited to, cations based on alkali metals or alkaline earth metals such as lithium, sodium, potassium, calcium, magnesium, and aluminum salts and the like, and nontoxic quaternary ammonium and amine cations including ammonium, tetramethylammonium, tetraethyl ammonium, methylammonium, dimethylammonium, trimethylammonium, ethylammonium, diethylammonium, triethylammonium, and the like. Other representative organic amines useful for the formation of base addition salts include ethylenediamine, ethanolamine, diethanolamine, piperidine, piperazine, and the like.

Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2nd ed., London (1988).

The formulation and route of administration chosen will be tailored to the individual subject, the nature of the condition to be treated in the subject, and generally, the judgment of the attending practitioner.

In some embodiments, the compounds of the invention are administered by injection most preferably by intravenous injection, but also by subcutaneous or intraperitoneal injection, and the like. Additional parenteral routes of administration include intramuscular and intraarticular injection. For intravenous or other parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The compounds may be administered as a single dose, a dose over time, as in i.v. or transdermal administration, or in multiple dosages. Dosages may be higher when the compounds are administered orally or transdermally as compared to, for example, i.v. administration.

Suitable dosage ranges for the compounds of the invention vary according to these considerations, but in general, the compounds are administered in the range of about 0.1 μg/kg-5 mg/kg of body weight; preferably the range is about 1 μg/kg-300 μg/kg of body weight; more preferably about 10 μg/kg-100 μg/kg of body weight. For a typical 70-kg human subject, thus, the dosage range is from about 0.7 μg-350 mg; preferably about 700 μg-21 mg; most preferably about 700 μg-10 mg. In certain embodiments, the compound is administered in the range of 5-15 mg/kg of body weight. In certain embodiments, the compound is administered at a dose of less than 11 mg/kg of body weight. In certain embodiments, the compound is administered at a dose of 10 mg/kg of body weight. In certain embodiments, suitable dosage is an amount between 1-500 mg. In certain embodiments, suitable dosage is an amount between 1-250 mg. In certain embodiments, suitable dosage is an amount between 1-100 mg. In certain embodiments, suitable dosage is an amount between 1-50 mg. In certain embodiments, suitable dosage is an amount between 1-25 mg. In certain embodiments, suitable dosage is an amount selected from the group consisting of 10 mg, 17 mg, 50 mg, 75 mg, 100 mg, 125 mg, 200 mg, 250 mg, and 400 mg. In certain embodiments, the suitable dosage is administered orally.

Compositions comprising a compound of the invention formulated in a pharmaceutically acceptable carrier can be prepared, placed in an appropriate container, and labeled for treatment of an indicated condition. Accordingly, there also is contemplated an article of manufacture, such as a container comprising a dosage form of a compound of the invention and a label containing instructions for use of the compound. Kits also are contemplated. For example, a kit can comprise a dosage form of a pharmaceutical composition and a package insert containing instructions for use of the composition in treatment of a medical condition. In either case, conditions indicated on the label can include treatment of an inflammatory condition.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

A group of items linked with the conjunction “or” should not be read as requiring mutual exclusivity among that group, but rather should also be read as “and/or” unless expressly stated otherwise. Although items, elements, or components of the invention may be described or claimed in the singular, the plural is contemplated to be within the scope thereof unless limitation to the singular is explicitly stated.

The following examples are offered to illustrate but not to limit the invention.

### EXAMPLES

### Example 1

Compounds of formula 1 and 2 can be made by methods known in the art. Specific examples of such compounds and some in vitro activity of these compounds are described in Chemistry & Biology, 17:123-34 (2010). Additional methods for making such compounds are described in U.S. Pat. No. 6,800,620.

The atropisomers of compounds 1 or 2 may be resolved by high-pressure liquid chromatography (HPLC) using a chiral phase normal or reversed phase column. Intermediate used to make 1 or 2 can also contain a mixture of atropisomers once the chiral bi-aryl linkage has been formed, and resolution of such intermediates by HPLC can also be carried out prior to completion of the synthesis of formula 1 or 2.

### Example 2

### Analytical HPLC Method Development for Separation of Atropisomers

This example describes the development of HPLC analytic methods for separating enantiomers of formula 1 or formula 2. In order to develop and optimize the separation of various atropisomers, a person having ordinary skill in the art can experiment with chromatographic parameters such as choice of column, mobile phase and flow rate. Methods for normal phase and reverse phase columns are described.

Normal phase. In this example, a racemic mixture of formula 2 is initially screened across a series of columns, such as CHIRALPAK®IA, IB, AD-H, AS and IC columns as well as CHIRALCEL®OD-H and OJ and using a suitable solvent system. When partial separation of atropisomers is observed on one of these chiral columns, further optimization of the separation is achieved by refining the solvent system to provide a baseline separation of atropisomers. When conditions for analytical separation have been determined, routine experimentation will typically translate into preparative separation conditions.

Reverse phase. A sample of a racemic mixture of formula 2 can alternatively be prepared in acetonitrile to be used to screen reverse phase HPLC separation conditions. The sample can be screened with CHIRALPAK® AD-RH®, AS-RH®, IB™, IC™, and CHIRALCEL®OJ-RH® columns, eluting with a suitable solvent. When partial separations is achieved with one such column, further optimization of the separation can be achieved with refinement of the solvent system using known methods and routine experimentation to provide baseline analytical separation. As discussed above, it is typically a matter of routine experimentation to then determine conditions for preparative separation of the atropisomers.

### Example 3

### Preparatory HPLC Separation of Atropisomers and Absolute Stereochemical Configuration

The absolute configuration of each isolated compound can been elucidated by x-ray crystallographic data.

### Example 4

### In Vitro Activity of 1, 2, 2(S) and 2(R)

Methods for determining and comparing activity of the separated atropisomers on individual isoforms of PI3K are well known in the art. For treatment of inflammation disorders, it is sometimes preferred to select a compound of formula 2 having low activity on the alpha and beta isoforms, and to select an inhibitor having high activity (low IC50) on at least the delta isoform and optionally having a gamma/delta IC50 ratio of less than about 10.

### Example 5

### Blood Plasma Concentration of 2(S) and 2(R) in Rats, Dogs and Humans

This example follows the concentration of formula 2(S) and 2(R) in the blood plasma or rat, dog and human subjects over time.

In order to perform the pharmacokinetic studies, formula 2 can be radiolabeled using ¹⁴C at the ortho-methyl group of the phenyl at position 3 of the quinazolinone ring. Radiolabeled 2:

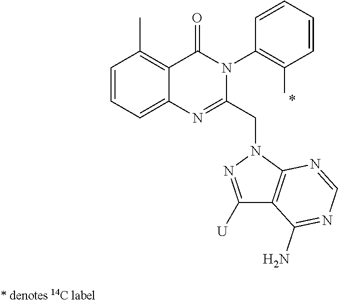

where U is as defined for formula 1.

The labeled racemic mixture or separated atropisomers can be administered to rats, dogs, and human subjects through oral or injection routes. The compounds can be dissolved in PEG 100 so any difference in dissolution rates would not play a role in the pharmacokinetic profile of the compounds. After administration of the compound, blood plasma of the subjects can be sampled over time and evaluated by analytical HPLC methods developed to identify and measure concentrations of formula 2(S) or 2(R) present in the sample. It may then be observed that the most abundant isomer measured in the plasma is formula 2(S), which accounts for more than half of the subject's exposure to formula 2.

The preceding examples and knowledge in the art enable one to practice the invention as described herein, and the invention is not limited to the examples. Additionally, any combination of embodiments described herein can be envisioned. Although individual features may be included in different claims, these may be advantageously combined. The following enumerated embodiments are exemplary embodiments contemplated to form part of the invention:

1. A composition comprising a compound of formula 1(S)

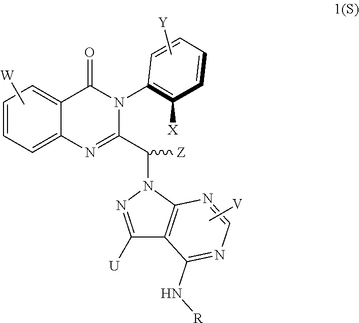

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

and wherein the compound of formula 1(S) is present in excess of its corresponding enantiomer of formula 1(R)

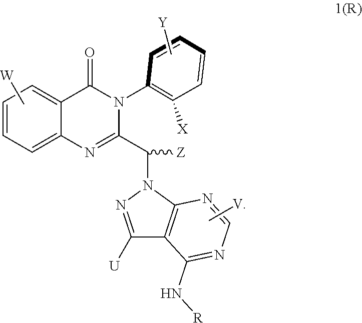

2. The composition according to embodiment 1 substantially free of the compound of formula 1(R).

3. The composition according to embodiment 1, wherein:
W is H, F, Cl, Me, or OMe;
Y is H or F;
X is Cl, Me or OMe;
V is H or Me; and
U is optionally substituted alkynyl or aryl.

4. The composition according to embodiment 1 or 2, wherein:
W is H, F, Cl, Me, or OMe;
Y is H or F;
X is Cl, Me or OMe;
V is H or Me; and
U is optionally substituted alkynyl or aryl.

5. A composition comprising a compound of formula 1(R)

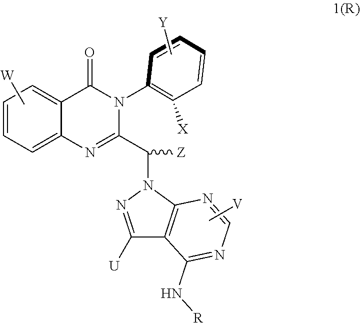

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

and wherein the compound of formula 1(R) is present in excess of the compound of formula 1(S)

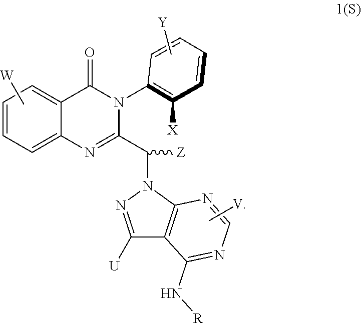

6. The composition according to embodiment 5 substantially free of its corresponding atropisomer of formula 1(S).

7. The composition according to embodiment 5, wherein
W is H, F, Cl, Me, or OMe;
Y is H or F;
X is Cl, Me or OMe;
V is H or Me; and
U is optionally substituted alkynyl or aryl.

8. The compound according to embodiment 5 or 6, wherein
W is H, F, Cl, Me, or OMe;
Y is H or F;
X is Cl, Me or OMe;
V is H or Me; and
U is optionally substituted alkynyl or aryl.

9. A composition comprising a compound of formula 2(S)

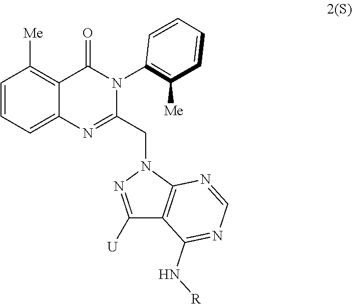

wherein U is selected from optionally substituted alkynyl and optionally substituted aryl; and R is H or C1-C4 acyl;

or a pharmaceutically acceptable salt thereof;

and wherein the compound of formula 2(S) is present in excess of the compound of formula 2(R)

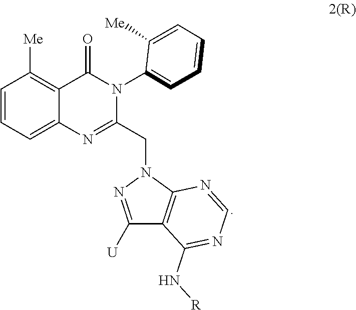

10. The compound according to embodiment 9 substantially free of the compound of formula 2(R).

11. A compound comprising an atropisomer of formula 2(R)

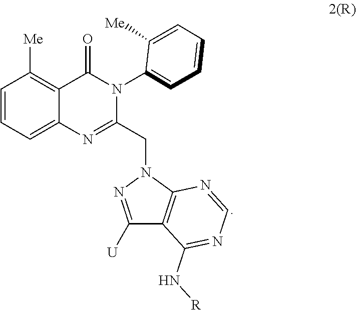

wherein U is selected from optionally substituted alkynyl and optionally substituted aryl;

and R is H or C1-C4 acyl;

or a pharmaceutically acceptable salt thereof;

and wherein the compound of formula 2(R) is present in excess of the compound of formula 2(S)

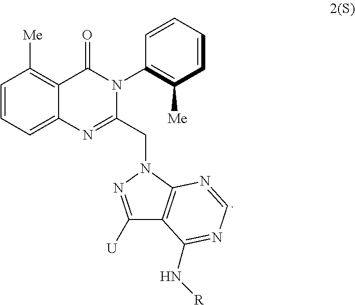

12. The compound according to embodiment 11 substantially free of the compound of formula 2(S).

13. A pharmaceutical composition comprising a compound according to embodiment 1, and a pharmaceutically acceptable carrier.

14. The pharmaceutical composition of embodiment 13, comprising a therapeutically effective amount of the optically active compound for the treatment of a condition selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

15. A pharmaceutical composition comprising the compound according to embodiment 5, and a pharmaceutically acceptable carrier.

16. The pharmaceutical composition of embodiment 15, comprising a therapeutically effective amount of the optically active compound for the treatment of a condition selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

17. A pharmaceutical composition comprising a compound according to any of embodiments 1-12, and a pharmaceutically acceptable carrier.

18. The pharmaceutical composition of embodiment 17, comprising a therapeutically effective amount of the optically active compound for the treatment of a condition selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

19. An optically active atropisomeric compound of formula 5:

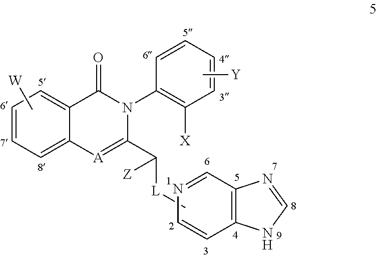

wherein A is CH or N;

W is an optional substituent that can be halo, C1-C4 alkyl, C1-C4 alkoxy, or CF₃;

Z can be H or C1-C4 alkyl, or if L is NR², Z and N can be linked together to form a 5-6 membered optionally substituted ring;

L can be NR² or S or a bond, and can be attached to position 6 or 9 of the purine ring;

Q can be H, Me, OMe, halo, or NH₂, or U, and is attached to the purine at position 2, 6, or 8 if L is attached at position 9, or at position 2 or 8 if L is attached at position 6;
U can be aryl, heteroaryl, cycloalkyl, heterocycloalkyl, alkyl, alkenyl, or alkynyl, each of which is optionally substituted;

X is Me, CF₃, Cl, CN, or Br;

Y can be H, C1-C4 alkyl, halo, CF₃, OMe, OH, NH₂, NHAc, or CN;

with the proviso that W and X are not both Me when Z is H, Q is NH₂, W is at position 5′ and L is a bond

or a pharmaceutically acceptable salt thereof.

20. The compound of embodiment 19, which is a compound of formula 5a or 5b:

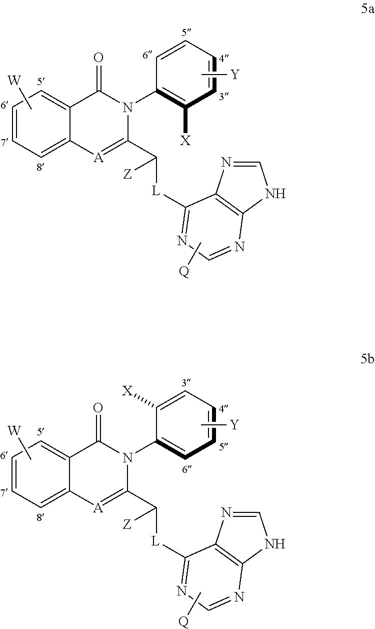

wherein A, Q, W, X, Y, Z and L are as defined in embodiment 19.

21. A compound of embodiment 19, which is a compound of formula 6a or 6b:

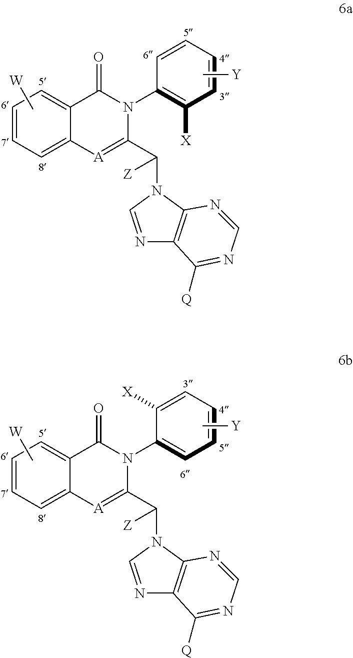

where A, Q, W, X, Y, Z and L are as defined in embodiment 19.

22. A compound of any of embodiments 19-21, wherein A is CH.

23. A compound of any of embodiments 19-21, wherein A is N.

24. A compound of any of embodiments 19-23, wherein X is Me.

25. A compound of any of embodiments 15-24, wherein Y is H.

26. A compound of any of embodiments 15-25, wherein W is H, F, Cl or Me and is located at position 5′ or 6′.

27. A compound of any of embodiments 15-26, wherein Q is H or NH₂.

28. A compound of embodiment 21, wherein Z is H.

29. A compound of embodiment 20, wherein Z is Me or Et.

30. A compound of embodiment 20, wherein L is NH.

31. A compound of any of embodiments 19-30, which is dextrorotatory.

32. A compound of any of embodiments 19-30, which is levorotatory.

33. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;

which comprises administering to said mammal a therapeutically effective amount of the optically active compound according to embodiment 1 or embodiment 19.

34. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;

which comprises administering to said mammal a therapeutically effective amount of the optically active compound according to embodiment 5, embodiment 20 or embodiment 21.

35. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;

which comprises administering to said mammal a therapeutically effective amount of the compound according to any of embodiments 1-12 or 19-32.

36. An optically active atropisomer obtained by chiral chromatographic separation of a mixture of the compounds of a compound of embodiment 1 or of embodiment 19.

37. The compound according to embodiment 21, wherein the predominant optically active atropisomer obtained is the compound of formula 6a substantially free of the compound of formula 6b.

38. The compound according to embodiment 21, wherein the predominant optically active atropisomer obtained is the compound of formula 6b substantially free of the compound of formula 6a.

39. An optically active atropisomer obtained by chiral chromatographic separation of an enantiomeric mixture of formula 1

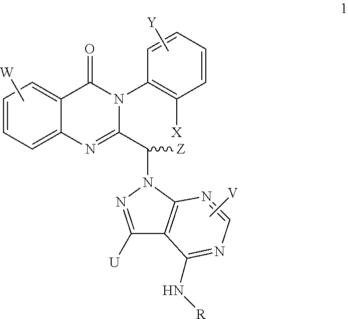

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

wherein an enantiomeric mixture of formula 1 is separated using a normal phase chiral column, and two peaks, A and B, are resolved,

wherein peak A and peak B represent atropisomers, 1(S) and 1(R), respectively,

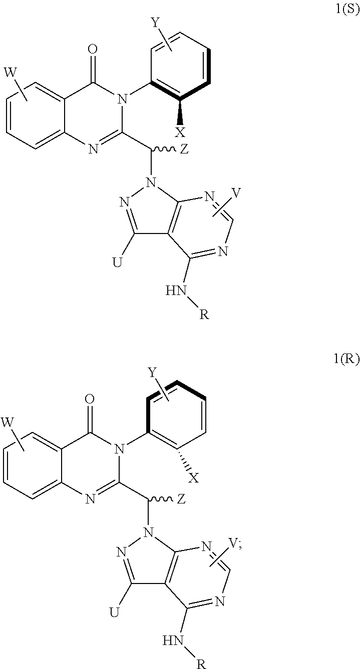

and

wherein the optically active atropisomer obtained is the second compound to elute from the column.

40. The compound according to embodiment 39, wherein the optically active atropisomer obtained is the compound of formula 1(S) substantially free of the compound of formula 1(R).

41. The compound according to embodiment 39, wherein the optically active atropisomer obtained is the compound of formula 1(R) substantially free of the compound of formula 1(S).

42. An optically active atropisomer obtained by chiral chromatographic separation of an enantiomeric mixture of formula 1

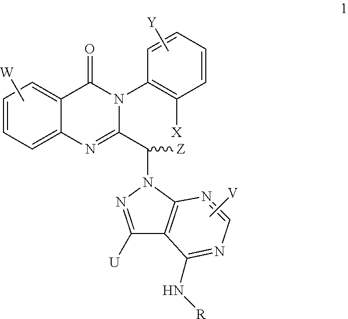

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

wherein an enantiomeric mixture of formula 1 is separated using a normal phase chiral column, and two peaks, A and B, are resolved,

wherein peak A and peak B represent atropisomers, 1(S) and 1(R), respectively,

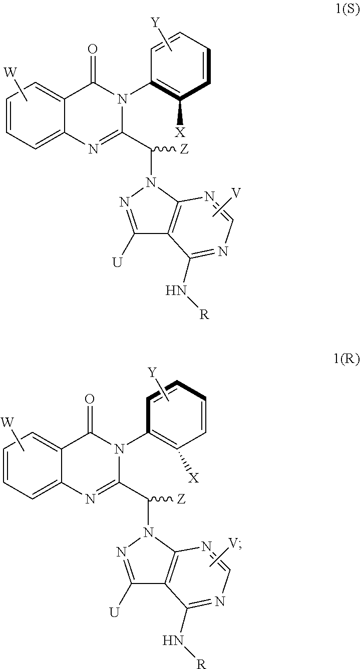

and

wherein the optically active atropisomer obtained is the first compound to elute from the column.

43. The compound according to embodiment 42, wherein the optically active atropisomer obtained is the compound of formula 1(S) substantially free of the compound of formula 1(R).

44. The compound according to embodiment 42, wherein the optically active atropisomer obtained is the compound of formula 1(R) substantially free of the compound of formula 1(S).

45. A compound of formula 1(S)

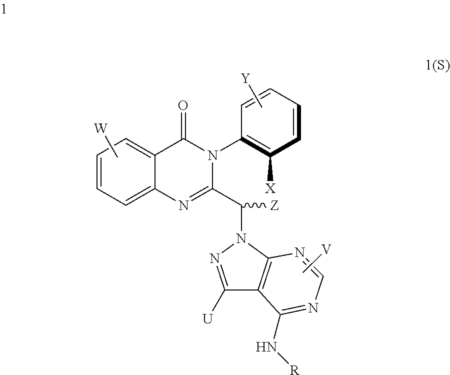

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof.

46. The compound according to embodiment 45, wherein:
W is H, F, Cl, Me, or OMe;
Y is H or F;
X is Cl, Me or OMe;
V is H or Me; and
U is optionally substituted alkynyl or aryl.

47. A compound comprising an atropisomer of formula 1(R)

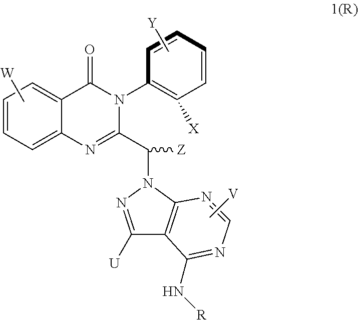

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof.

48. The compound according to embodiment 47, wherein
W is H, F, Cl, Me, or OMe;
Y is H or F;
X is Cl, Me or OMe;
V is H or Me; and
U is optionally substituted alkynyl or aryl.

49. A compound comprising an atropisomer of formula 2(S)

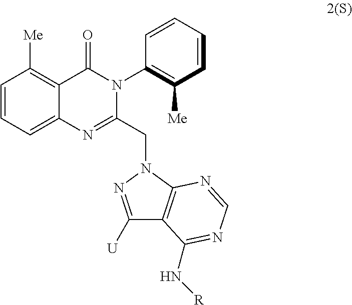

wherein U is selected from optionally substituted alkynyl and optionally substituted aryl; and R is H or C1-C4 acyl;

or a pharmaceutically acceptable salt thereof.

50. A compound comprising an atropisomer of formula 2(R)

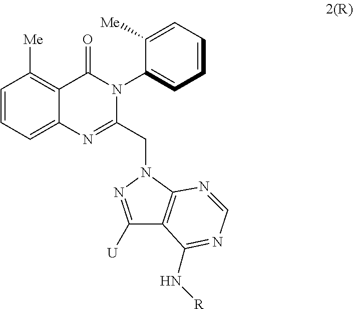

wherein U is selected from optionally substituted alkynyl and optionally substituted aryl;

and R is H or C1-C4 acyl;

or a pharmaceutically acceptable salt thereof.

51. A composition comprising an optically active compound according to embodiment 45 or 46, and a pharmaceutically acceptable carrier.

52. The composition of embodiment 51, wherein the compound of formula 1(S) is present in excess of the compound of formula 1(R)

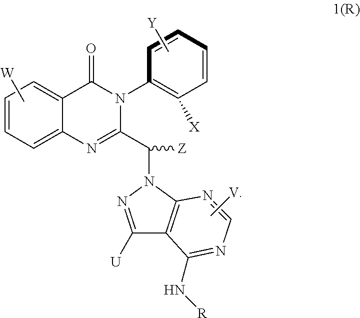

53. The composition of embodiment 51 substantially free of the compound of formula 1(R).

54. A composition comprising an optically active compound according to embodiment 47 or 48, and a pharmaceutically acceptable carrier.

55. The composition of embodiment 54, wherein the compound of formula 1(R) is present in excess of the compound of formula 1(S)

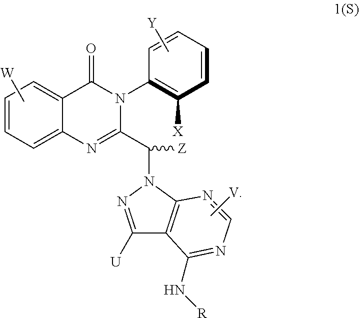

56. The composition of embodiment 54 substantially free of the compound of formula 1(S).

57. A composition comprising the optically active compound according to embodiment 49, and a pharmaceutically acceptable carrier.

58. The composition of embodiment 57, wherein the compound of formula 2(S) is present in excess of the compound of formula 2(R)

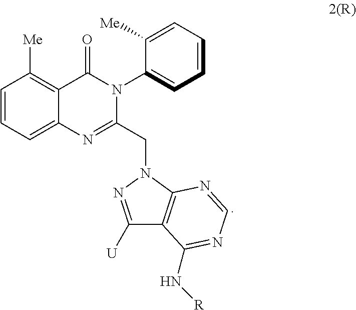

59. The composition of embodiment 57 substantially free of the compound of formula 2(R).

60. A composition comprising the optically active compound according to embodiment 50, and a pharmaceutically acceptable carrier.

61. The composition of embodiment 60, wherein the compound of formula 2(R) is present in excess of the compound of formula 2(S)

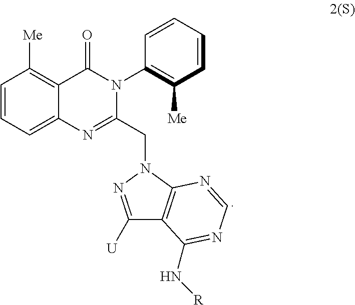

62. The composition of embodiment 60 substantially free of the compound of formula 2(S).

63. The composition of any of embodiments 51-62, comprising a therapeutically effective amount of the optically active compound for the treatment of a condition selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury.

64. An optically active atropisomeric compound of formula 5:

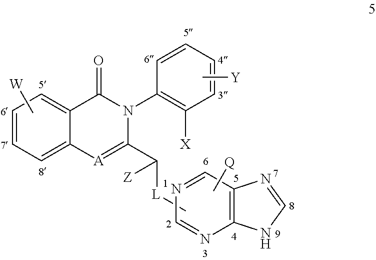

wherein A is CH or N;

W is an optional substituent that can be halo, C1-C4 alkyl, C1-C4 alkoxy, or CF₃;

Z can be H or C1-C4 alkyl, or if L is NR², Z and N can be linked together to form a 5-6 membered optionally substituted ring;

L can be NR² or S or a bond, and can be attached to position 6 or 9 of the purine ring;

Q can be H, Me, OMe, halo, or NH₂ or U, and is attached to the purine at position 2, 6, or 8 if L is attached at position 9, or at position 2 or 8 if L is attached at position 6;
U can be aryl, heteroaryl, cycloalkyl, heterocycloalkyl, alkyl, alkenyl, or alkynyl, each of which is optionally substituted;

X is Me, CF₃, Cl, CN, or Br;

Y can be H, C1-C4 alkyl, halo, CF₃, OMe, OH, NH₂, NHAc, or CN;

with the proviso that W and X are not both Me when Z is H, Q is NH₂, W is at position 5′ and L is a bond

or a pharmaceutically acceptable salt thereof.

65. The compound of embodiment 64, which is a compound of formula 5a or 5b:

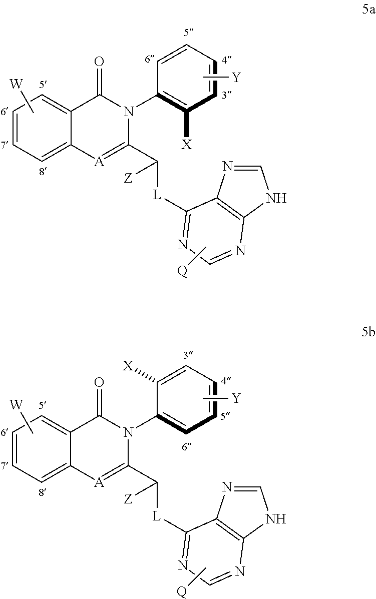

wherein A, Q, W, X, Y, Z and L are as defined in embodiment 20.

66. A compound of embodiment 64, which is a compound of formula 6a or 6b:

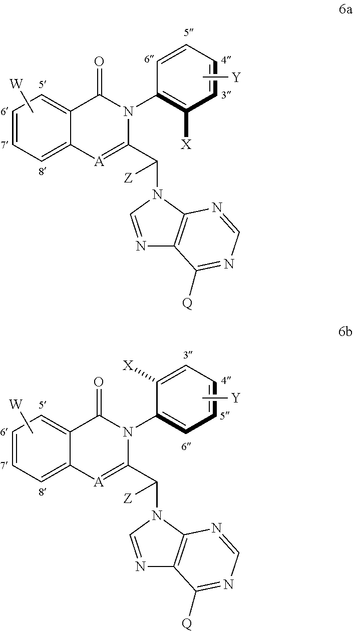

where A, Q, W, X, Y, Z and L are as defined in embodiment 64.

67. A compound of any of embodiments 64-66, wherein A is CH.

68. A compound of any of embodiments 64-66, wherein A is N.

69. A compound of any of embodiments 64-68, wherein X is Me.

70. A compound of any of embodiments 64-69, wherein Y is H.

71. A compound of any of embodiments 64-70, wherein W is H, F, Cl or Me and is located at position 5′ or 6′.

72. A compound of any of embodiments 64-71, wherein Q is H or NH2.

73. A compound of embodiment 66, wherein Z is H.

74. A compound of embodiment 65, wherein Z is Me or Et.

75. A compound of embodiment 65, wherein L is NH.

76. A compound of any of embodiments 64-75, which is dextrorotatory.

77. A compound of any of embodiments 64-75, which is levorotatory.

78. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;

which comprises administering to said mammal a therapeutically effective amount of the optically active compound according to any of embodiments 45-50 or 46-77.

79. An optically active atropisomer obtained by chiral chromatographic separation of a mixture of the compounds of a compound according to any of embodiments 45-50 or 64-77.

80. A composition comprising the optically active compound according to embodiment 79, and a pharmaceutically acceptable carrier.

81. The composition according to embodiment 80, wherein the predominant optically active atropisomer obtained is the compound of formula 6a substantially free of the compound of formula 6b.

82. The composition according to embodiment 80, wherein the predominant optically active atropisomer obtained is the compound of formula 6b substantially free of the compound of formula 6a.

83. An optically active atropisomer obtained by chiral chromatographic separation of an enantiomeric mixture of formula 1

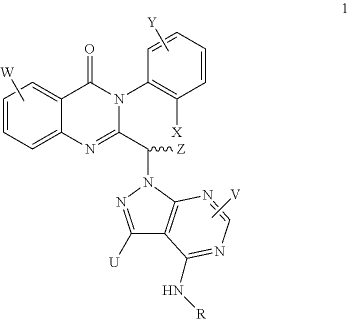

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

wherein an enantiomeric mixture of formula 1 is separated using a normal phase chiral column, and two peaks, A and B, are resolved,

wherein peak A and peak B represent atropisomers, 1(S) and 1(R), respectively,

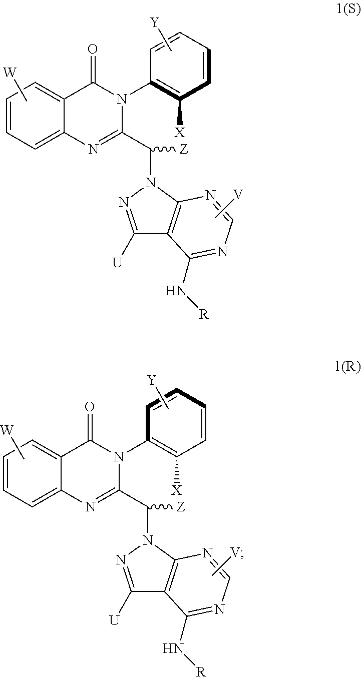

and

wherein the optically active atropisomer obtained is the second compound to elute from the column.

84. A composition comprising the optically active compound according to embodiment 83.

85. The composition according to embodiment 84, wherein the optically active atropisomer obtained is the compound of formula 1(S) substantially free of the compound of formula 1(R).

86. The compound according to embodiment 84, wherein the optically active atropisomer obtained is the compound of formula 1(R) substantially free of the compound of formula 1(S).

87. An optically active atropisomer obtained by chiral chromatographic separation of an enantiomeric mixture of formula 1

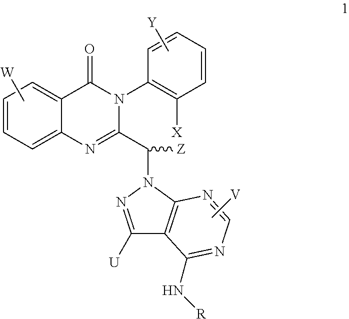

wherein W, Y, and V are each independently H, halo, R¹, OR¹, CF₃, CN, where each R¹ is independently H or C1-C4 alkyl;

X is selected from Cl, Me, CF₃, CN, and OMe;

Z is H, Me, Et, n-Pr, or cyclopropyl;

R is H or C1-C4 acyl; and

U is selected from aryl, alkenyl, and alkynyl, each of which is optionally substituted;

or a pharmaceutically acceptable salt thereof;

wherein an enantiomeric mixture of formula 1 is separated using a normal phase chiral column, and two peaks, A and B, are resolved,

wherein peak A and peak B represent atropisomers, 1(S) and 1(R), respectively,

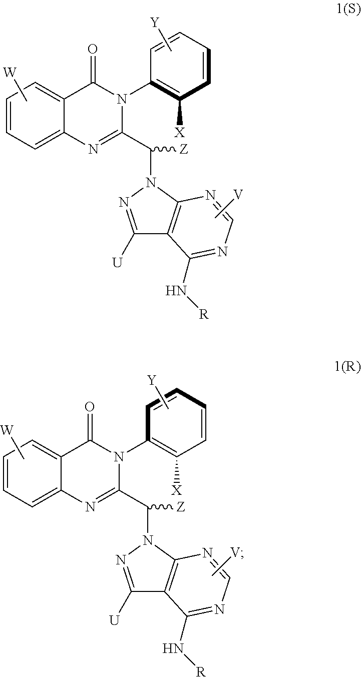

and

wherein the optically active atropisomer obtained is the first compound to elute from the column.

88. A composition comprising the optically active compound according to embodiment 87.

89. The composition according to embodiment 88, wherein the optically active atropisomer obtained is the compound of formula 1(S) substantially free of the compound of formula 1(R).

90. The composition according to embodiment 88, wherein the optically active atropisomer obtained is the compound of formula 1(R) substantially free of the compound of formula 1(S).

91. The composition of any of claims 51-62, comprising a therapeutically effective amount of the compound for the treatment of cancer.

92. A method of treating a condition in a mammal, wherein the condition is cancer, which comprises administering to said mammal a therapeutically effective amount of the compound according to any of claims 45-50 or 64-77.

1-48. (canceled)

49. A compound of formula 1(S) or 1(R) 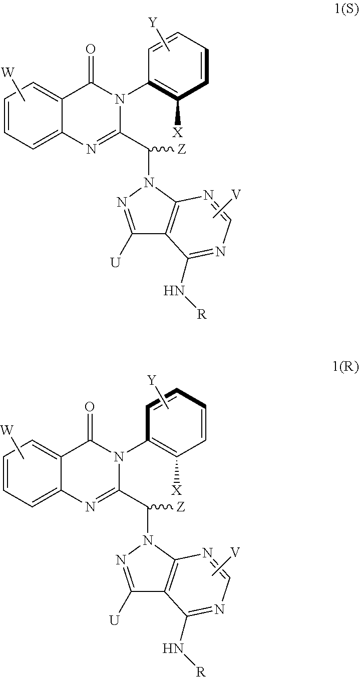
wherein W, Y, and V are each independently selected from the group consisting of H, halo, R¹, OR¹, CF₃, and CN,
each R¹ is independently H or C1-C4 alkyl;
X is selected from the group consisting of Cl, Me, CF₃, CN, and OMe;
Z is selected from the group consisting of H, Me, Et, n-Pr, and cyclopropyl;
R is H or C1-C4 acyl; and
U is selected from the group consisting of aryl, alkenyl, and alkynyl, each of which is optionally substituted;
or a pharmaceutically acceptable salt thereof.

50. The compound according to claim 49, wherein:
W is selected from the group consisting of H, F, Cl, Me, and OMe;
Y is H or F;
X is selected from the group consisting of Cl, Me and OMe;
V is H or Me; and
U is alkynyl or aryl, each of which is optionally substituted,
or a pharmaceutically acceptable salt thereof.

51. The compound of claim 49, wherein the compound is a compound of formula 2(S) 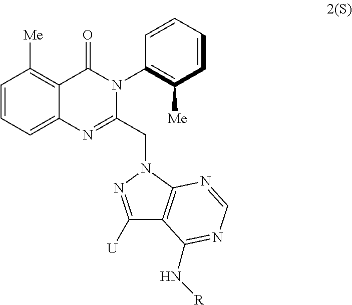
wherein U is selected from optionally substituted alkynyl and optionally substituted aryl; and
R is H or C1-C4 acyl;
or a pharmaceutically acceptable salt thereof.

52. The compound of claim 49, wherein the compound is a compound of formula 2(R) 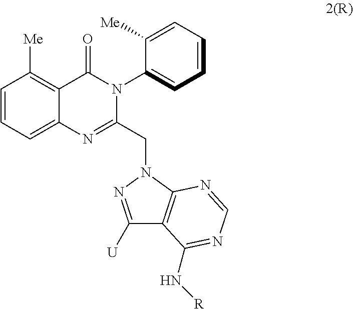
wherein U is selected from optionally substituted alkynyl and optionally substituted aryl;
and R is H or C1-C4 acyl;
or a pharmaceutically acceptable salt thereof.

53. A composition comprising a compound according to claim 49, and a pharmaceutically acceptable carrier.

54. An optically active atropisomeric compound of formula 5: 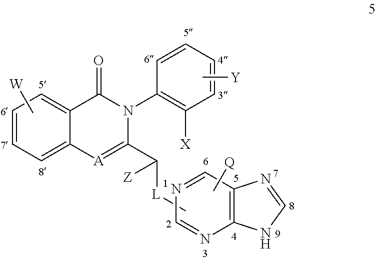
wherein A is CH or N;
W is an optional substituent selected from the group consisting of halo, C1-C4 alkyl, C1-C4 alkoxy, and CF₃;
Z is H or C1-C4 alkyl, or
if L is NR², Z and N can be linked together to form a 5-6 membered optionally substituted ring;
L is selected from the group consisting of NR², S, and a bond, and can be attached to position 6 or 9 of the purine ring;
Q is selected from the group consisting of H, Me, OMe, halo, NH₂, and U, and is attached to the purine at position 2, 6, or 8 if L is attached at position 9, or at position 2 or 8 if L is attached at position 6;
U is selected from the group consisting of aryl, heteroaryl, cycloalkyl, heterocycloalkyl, alkyl, alkenyl, and alkynyl, each of which is optionally substituted;
X is selected from the group consisting of Me, CF₃, Cl, CN, and Br;
Y is selected from the group consisting of H, C1-C4 alkyl, halo, CF₃, OMe, OH, NH₂, NHAc, and CN;
with the proviso that W and X are not both Me when Z is H, Q is NH₂, W is at position 5′ and L is a bond,
or a pharmaceutically acceptable salt thereof.

55. The optically active atropisomeric compound of claim 54, which is a compound of formula 5a or 5b: 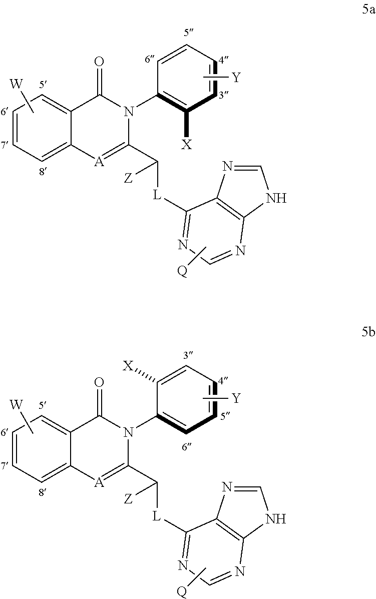
or pharmaceutically acceptable salt thereof,
wherein A, Q, W, X, Y, Z and L are as defined in claim 54.

56. A composition comprising a compound according to claim 54, and a pharmaceutically acceptable carrier.

57. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;
which comprises administering to said mammal a therapeutically effective amount of the compound according to claim 49.

58. A method of treating a condition in a mammal, wherein the condition is selected from the group consisting of chronic inflammatory diseases, tissue or organ transplant rejections, graft versus host disease (GVHD), multiple organ injury syndromes, acute glomerulonephritis, reactive arthritis, hereditary emphysema, chronic obstructive pulmonary disease (COPD), cystic fibrosis, adult respiratory distress syndrome (ARDS), ischemic-reperfusion injury, stroke, rheumatoid arthritis (RA), osteoarthritis (OA), asthma, allergic rhinitis, lupus nephritis, Crohn's disease, ulcerative colitis, necrotizing enterocolitis, pancreatitis, Pneumocystis carinii pneumonia (PCP), inflammatory bowel disease (IBD), severe acute respiratory syndrome (SARS), sepsis, community acquired pneumonia (CAP), multiple sclerosis (MS), myocardial infarction, respiratory syncytial virus (RSV) infection, dermatitis, acute purulent meningitis, thermal injury, granulocyte transfusion associated syndromes, cytokine-induced toxicity, and spinal cord injury;
which comprises administering to said mammal a therapeutically effective amount of the compound according to claim 54.

59. An optically active atropisomer obtained by chiral chromatographic separation of an enantiomeric mixture of formula 1 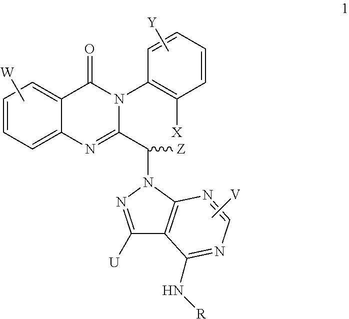
wherein W, Y, and V are each independently selected from the group consisting of H, halo, R¹, OR¹, CF₃, and CN,
each R¹ is independently H or C1-C4 alkyl;
X is selected from the group consisting of Cl, Me, CF₃, CN, and OMe;
Z is selected from the group consisting of H, Me, Et, n-Pr, and cyclopropyl;
R is H or C1-C4 acyl; and
U is selected from the group consisting of aryl, alkenyl, and alkynyl, each of which is optionally substituted;
or a pharmaceutically acceptable salt thereof;
wherein an enantiomeric mixture of formula 1 is separated using a normal phase chiral column, and two peaks, A and B, are resolved,
wherein peak A and peak B represent atropisomers, 1(S) and 1(R), respectively, 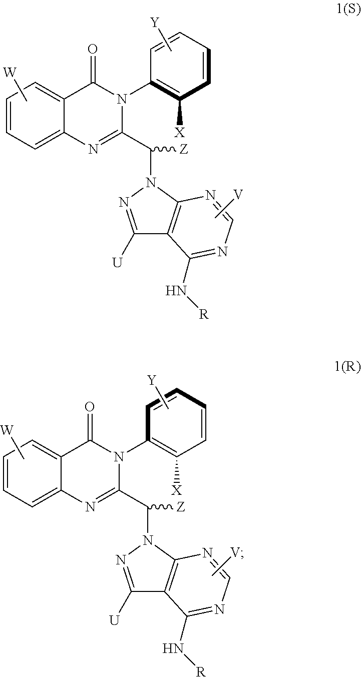
and
wherein the optically active atropisomer obtained is the first or second compound to elute from the column.

60. A composition comprising the optically active atropisomer or pharmaceutically acceptable salt thereof according to claim 59.

61. The composition according to claim 60, wherein the optically active atropisomer obtained is the compound of formula 1(S) or pharmaceutically acceptable salt thereof substantially free of the compound of formula 1(R) or pharmaceutically acceptable salt thereof.

62. The composition according to claim 60, wherein the optically active atropisomer obtained is the compound of formula 1(R) or pharmaceutically acceptable salt thereof substantially free of the compound of formula 1(S) or pharmaceutically acceptable salt thereof.

63. A method of treating a condition in a mammal, wherein the condition is cancer, which comprises administering to said mammal a therapeutically effective amount of the compound according claim 49.

64. A method of treating a condition in a mammal, wherein the condition is cancer, which comprises administering to said mammal a therapeutically effective amount of the compound according to claim 54.

65. A method of treating a condition in a mammal, wherein the condition is cancer, which comprises administering to said mammal a therapeutically effective amount of the compound according to claim 59.
